SEQUENCE LISTING (30 sequences)

SEQ ID NO: 1 - DNA, 16 bp, synthetic construct
  source 1..16: mol_type = other DNA
cccaacactccacacc

SEQ ID NO: 2 - DNA, 20 bp, synthetic construct
  source 1..20: mol_type = other DNA
tcttaggagctgtccgaggg

SEQ ID NO: 3 - DNA, 27 bp, synthetic construct
  source 1..27: mol_type = other DNA
cacacactacacacacccacccgtctc

SEQ ID NO: 4 - DNA, 22 bp, synthetic construct
  source 1..22: mol_type = other DNA
cgttttatttgggaggagcgac

SEQ ID NO: 5 - DNA, 25 bp, synthetic construct
  source 1..25: mol_type = other DNA
accactatactttacgaaccctacg

SEQ ID NO: 6 - DNA, 28 bp, synthetic construct
  source 1..28: mol_type = other DNA
gaataatgttttatttgggaggagtgat

SEQ ID NO: 7 - DNA, 27 bp, synthetic construct
  source 1..27: mol_type = other DNA
aaaccactatactttacaaaccctaca

SEQ ID NO: 8 - DNA, 53 bp, synthetic construct
  source 1..53: mol_type = other DNA
tgtaaaacgacggccagtgttgagaggttagtgttttaaatatgtattttagg

SEQ ID NO: 9 - DNA, 39 bp, synthetic construct
  source 1..39: mol_type = other DNA
caggaaacagctatgacctaaaacccctttacccaaccc

SEQ ID NO: 10 - DNA, 125 bp, synthetic construct
  source 1..125: mol_type = other DNA
accttgttggcgggagaaggaataacgttttatttgggaggagcgacggattatagccaataagagagctcaagacgtagggttcgtaaagtatagtggtttcgtgggaccttagaggtggagca

SEQ ID NO: 11 - DNA, 125 bp, synthetic construct
  source 1..125: mol_type = other DNA
accttgttggcgggagaaggaataatgttttatttgggaggagtgatggattatagccaataagagagctcaagatgtagggtttgtaaagtatagtggtttcgtgggaccttagaggtggagca

SEQ ID NO: 12 - DNA, 23 bp, synthetic construct
  source 1..23: mol_type = other DNA
ggagaaggaataacgccttatct

SEQ ID NO: 13 - DNA, 20 bp, synthetic construct
  source 1..20: mol_type = other DNA
ccactatactttgcgagccc

SEQ ID NO: 14 - DNA, 46 bp, synthetic construct
  source 1..46: mol_type = other DNA
tgtaaaacgacggccagttgttataagattattaagttggtgtaaa

SEQ ID NO: 15 - DNA, 46 bp, synthetic construct
  source 1..46: mol_type = other DNA
caggaaacagctatgacctttacaacaaaacacaaaatttttataa

SEQ ID NO: 16 - DNA, 83 bp, synthetic construct
  source 1..83: mol_type = other DNA
gaataacgttttatttgggaggagcgacggattatagttaataagagagtttaagacgtagggttcgtaaagtatagtggttt

SEQ ID NO: 17 - DNA, 33 bp, synthetic construct
  source 1..33: mol_type = other DNA
ggtataacgttttatttgggaggagcgacggat

SEQ ID NO: 18 - protein, 11 aa, synthetic construct
RYNVLFGRSDG

SEQ ID NO: 19 - DNA, 33 bp, synthetic construct
  source 1..33: mol_type = other DNA
ggaataatgttttatttgggaggagtgatggat

SEQ ID NO: 20 - protein, 11 aa, synthetic construct
RNNVLFGRSDG

SEQ ID NO: 21 - DNA, 25 bp, synthetic construct
  source 1..25: mol_type = other DNA
gcatcccaagcatttcatatcacca

SEQ ID NO: 22 - DNA, 26 bp, synthetic construct
  source 1..26: mol_type = other DNA
gacgtagggttcgtaaagtatagtgg

SEQ ID NO: 23 - protein, 6 aa, synthetic construct
TGSSIV

SEQ ID NO: 24 - DNA, 26 bp, synthetic construct
  source 1..26: mol_type = other DNA
gatgtagggtttgtaaagtatagtgg

SEQ ID NO: 25 - protein, 6 aa, synthetic construct
MGLSIV

SEQ ID NO: 26 - DNA, 23 bp, synthetic construct
  source 1..23: mol_type = other DNA
cgttttatttgggaggaggcgac

SEQ ID NO: 27 - DNA, 34 bp, synthetic construct
  source 1..34: mol_type = other DNA
ggtataacgttttatttgggaggagcgacggatt

SEQ ID NO: 28 - protein, 12 aa, synthetic construct
RYNVLFGRSDGL

SEQ ID NO: 29 - DNA, 31 bp, synthetic construct
  source 1..31: mol_type = other DNA
aagacgtagggttcgtaaagtatagtggttt

SEQ ID NO: 30 - protein, 8 aa, synthetic construct
KTGSSIVV

(19) United States
(12) Patent Application Publication
(10) Pub. No.: US 2026/0117327 A1
(43) Pub. Date: Apr. 30, 2026

(54) EARLY DIAGNOSIS OF CANCER THROUGH EBV PROMOTER METHYLATION

(71) Applicant: THE JOHNS HOPKINS UNIVERSITY, Baltimore, MD (US)

(72) Inventors: Richard Ambinder, Baltimore, MD (US); Rena Xian, Baltimore, MD (US); Logan George, Baltimore, MD (US)

(21) Appl. No.: 19/374,484

(22) Filed: Oct. 30, 2025

Related U.S. Application Data
(60) Provisional application No. 63/714,692, filed on Oct. 31, 2024.

(51) Int. Cl. C12Q 1/70; C12Q 1/6886

(52) U.S. Cl. CPC C12Q 1/701; C12Q 1/6886; C12Q 2600/154

(57) ABSTRACT

The present disclosure provides methods for distinguishing virion DNA from tumor-derived viral DNA within viral nucleic acid sequences by determining a DNA methylation status of the viral nucleic acid sequence. The methods are used for diagnosing and treating cancer. The method of diagnosing a subject with cancer or treating cancer in a subject comprises identifying a viral nucleic acid sequence in a sample as virion DNA or tumor-derived viral DNA including determining a DNA methylation status of the viral nucleic acid sequence. The methylation of the viral nucleic acid sequence is indicative of tumor-derived viral DNA and the absence of methylation of the viral nucleic acid sequence is indicative of virion DNA.

### CROSS-REFERENCE TO RELATED APPLICATIONS

### Background of the Invention

### Field of the Invention

This application claims benefit of priority under 35 U.S.C. § 119(e) of U.S. Provisional Application No. 63/714,692, filed Oct. 31, 2024. The disclosure of the prior application is considered part of and is herein incorporated by reference in the disclosure of this application in its entirety.

### STATEMENT REGARDING GOVERNMENT FUNDING

This invention was made with government support under grants CA006973, CA250069, and CA244811 awarded by the National Institutes of Health. The government has certain rights in the invention.

### INCORPORATION OF SEQUENCE LISTING

The material in the accompanying sequence listing is hereby incorporated by reference into this application. The accompanying sequence listing xml file, name JHU4790-1, was created on Sep. 19, 2025 and is 27,870 bytes in size.

### BACKGROUND OF THE INVENTION

### Field of the Invention

The present invention relates to a method of diagnosing cancer, and more specifically to diagnosing EBV related cancer by analyzing the epigenetic modulation statute of EBV DNA.

### Background Information

Epstein-Barr virus (EBV) is a ubiquitous pathogen that infects >90% of the world's population. Following a primary infection, EBV maintains a latent reservoir predominantly in memory B cells, and rarely, T cells and NK cells. EBV is also able to infect epithelial cells. While the majority of these latent infections are benign, particularly in healthy individuals, latently EBV-infected cells may give rise to cancer, establishing EBV as a carcinogen. These cancers may arise in immunocompromised individuals, such as people living with HIV, in the elderly, or those living in regions where EBV-associated (EBV(+)) cancers are endemic. These tumors include Burkitt lymphoma, diffuse large B cell lymphoma, Hodgkin lymphoma, NK/T cell lymphoma, gastric carcinoma, and nasopharyngeal carcinoma.

Following primary infection of the host, EBV establishes life-long latent infection. Cells may be latently infected (virions are not produced, and genomes are maintained across cell divisions) or lytically infected (with production of virions). EBV latency can be described as a series of programs, called Latency I, II, and III, which range from more to less restrictive expression of latency genes, respectively. Within each latency program, different combinations of latency genes are expressed. Some EBV latency genes are required for B cells immortalization in vitro, such as EBNA2; however, expression of these viral oncogenes does not necessarily correlate with oncogenesis. While EBV-associated oncogenesis is still not fully understood, it is known that different tumor types are frequently associated with specific latency programs.

EBV latency programming and lytic reactivation are coordinated through a sequence of epigenetic modifications to the viral genome, including DNA methylation at viral latency promoters. Three of these latency promoters are the C promoter (Cp), the W promoter (Wp) and the Q promoter (Qp); expression from or methylation of these promoters allows EBV to epigenetically modulate expression of the EBNA family of proteins. Wp is located within the BamHI W repeats, also known as IR1, and Cp is just upstream of IR1 in a non-repetitive region. Expression from Cp is characteristic of the Latency III program, which is found in in vitro immortalized lymphoblastoid cells (LCLs) and in some lymphoproliferative disorders. Latency I expression is the most restrictive program, with Cp and Wp methylated. Latency I is associated with Burkitt lymphoma and gastric cancer. Latency I EBV genomes are densely methylated, with only EBNA1 expressed from Qp. Latency II, where only EBNA1 and LMP1/2 are expressed, is associated with nasopharyngeal carcinoma and Hodgkin lymphoma.

EBV genomes packaged into virions are devoid of methylation. Methylated EBV genomes must have been synthesized in latently infected cells. Thus, the state of methylation provides some insights into the source of DNA in a specimen. Previous investigations have used bisulfite sequencing and methyl-binding protein (MBD) assays to explore methylation of EBV circulating cell free DNA (cfDNA) as a cancer biomarker. Despite their power, these methods face some limitations. Bisulfite sequencing is costly and requires specialized equipment. MBD assays target regions of methylation rather than particular sites, and thus the results may be more ambiguous. Both methods require large amounts of input DNA, which may be challenging to achieve in low-copy samples. There is a need for methods for assaying EBV cfDNA methylation.

### SUMMARY OF DISCLOSURE

The present disclosure is based on the seminal discovery that quantitative methylation-specific PCR for the C Promoter (MSPCP) can sensitively detect, quantify, and differentiate virion-derived and cell/tumor-derived EBV DNA. The present invention is exemplified but not limited by the disclosure in the Examples herein as well as Exhibit A, Exhibit B, Exhibit C, and Exhibit D, which are herein incorporated by reference in their entireties.

In certain embodiments, the present disclosure provides a method of identifying a viral nucleic acid sequence in a sample as virion DNA or tumor-derived viral DNA including determining a DNA methylation status of the viral nucleic acid sequence, wherein methylation of the viral nucleic acid sequence is indicative of tumor-derived viral DNA and the absence of methylation of the viral nucleic acid sequence is indicative of virion DNA, thereby identifying the viral nucleic acid sequence as virion DNA or tumor-derived viral DNA.

In some aspects, the viral nucleic acid sequence is an Epstein-Barr virus (EBV) nucleic acid sequence. In some aspects, the EBV nucleic acid sequence includes an EBV C promoter (Cp), an EBV W promoter (Wp), EBV Q promoter (Qp), or an EBV F promoter (Fp). In some aspects, EBV nucleic acid sequence includes an EBV Cp. In some aspects, determining the DNA methylation status of the viral nucleic acid sequence includes determining the methylation status of EBV Cp. In some aspects, determining the DNA methylation status of EBV Cp includes performing a methylation specific PCR. In some aspects, performing the methylation specific PCR includes performing a qPCR. In some aspects, performing the methylation specific PCR includes using primers targeting CpG islands in EBV Cp. In some aspects, the CpG islands are located within coordinates 11038-11117 of EBV Type 1 reference genome NC_007605.1. In some aspects, the primers include: a) methylated-CpG targeting (M) primers and b) unmethylated-CpG targeting (U) primers. In some aspects, the M primers include a nucleic acid sequence including SEQ ID NO:4 and SEQ ID NO:5. In some aspects, the U primers include a nucleic acid sequence including SEQ ID NO:6 and SEQ ID NO:7. In one aspect, a methylated status of the viral DNA is indicative of presence of tumor-derived viral DNA and/or tumor-derived viral DNA in the sample. In another aspect, an unmethylated status of the viral DNA is indicative of absence of tumor-derived viral DNA and/or tumor-derived viral DNA in the sample. In some aspects, the sample includes less than about 300 EBV copy number. In some aspects, the sample includes less than about 100 EBV copy number. In some aspects, the sample includes about 50 EBV copy number. In some aspects, the sample is a tissue biopsy, plasma, saliva, tumor sample and/or blood sample. In one aspect, the sample is a plasma sample. In another aspect, the sample is a whole saliva sample.

In certain embodiments, the present disclosure provides a method of diagnosing cancer in a subject comprising identifying a viral nucleic acid sequence as tumor-derived viral DNA in a sample from the subject, wherein identifying the viral nucleic acid sequence as tumor-derived viral DNA includes determining a DNA methylation status of the viral nucleic acid sequence, wherein detecting methylated tumor-derived viral DNA is indicative of presence of cancer is the subject, thereby diagnosing the subject with cancer.

In some aspects, determining the DNA methylation status of the viral nucleic acid sequence comprises determining the methylation status of EBV Cp and/or EBV Wp. In some aspects, the DNA methylation status is indicative of an EBV latency program. In some aspects, the EBV latency program is type I latency program, type II latency program, or type III latency program. In one aspect, a methylated status of the viral DNA is indicative of presence of tumor-derived viral DNA in the sample. In another aspect, an unmethylated status of the viral DNA is indicative of absence of tumor-derived viral DNA in the sample. In some aspects, the viral nucleic acid sequence includes cell free DNA (cfDNA). In some aspects, the cancer is an EBV-related cancer. In some aspects, the EBV-related cancer is a blood cancer or an epithelial cancer. In some aspects, the blood cancer is Burkitt lymphoma, diffuse large B cell lymphoma, Hodgkin lymphoma, or NK/T cell lymphoma. In some aspects, the epithelial cancer is gastric carcinoma, or nasopharyngeal carcinoma. In some aspects, the subject is a mammal. In some aspects, the mammal is human.

In certain embodiments, the present disclosure provides a kit including: a) methylated-CpG targeting (M) primers, wherein the M primers include a nucleic acid sequence comprising SEQ ID NO:4 and SEQ ID NO:5, b) unmethylated-CpG targeting (U) primers, wherein the U primers include a nucleic acid sequence comprising SEQ ID NO:6 and SEQ ID NO:7, and c) instructions for using the U and M primers for methylation specific PCR on a biological sample.

In further aspects, the kit includes a sample collection device for obtaining the biological sample from a subject. In some aspects, the presence of methylation in the sample after PCR is indicative of an EBV-related cancer. In some aspects, the sample includes viral nucleic acid sequences. In some aspects, the viral nucleic acid sequences include cfDNA or cell-associated DNA. In some aspects, the EBV-related cancer is a blood cancer or an epithelial cancer. In some aspects, the blood cancer is Burkitt lymphoma, diffuse large B cell lymphoma, Hodgkin lymphoma, or NK/T cell lymphoma.

In certain embodiments, the present disclosure provides a method of treating cancer in a subject in need thereof including: a) identifying a viral nucleic acid sequence as tumor-derived viral DNA in a sample from the subject, wherein identifying the viral nucleic acid sequence as tumor-derived viral DNA includes determining a DNA methylation status of the viral nucleic acid sequence, wherein detecting methylated tumor-derived viral DNA is indicative of presence of cancer is the subject and wherein an unmethylated status of the viral DNA is indicative of absence of tumor-derived viral DNA in the sample; and b) administering an anti-cancer treatment to the subject diagnosed with cancer, thereby treating the cancer in the subject.

In some aspects, the anti-cancer treatment is chemotherapy, radiation therapy, immunotherapy, resection surgery, hormone therapy, stem cell or bone marrow transplant, or a combination thereof. In some aspects, a methylated status of the viral DNA is indicative of presence of tumor-derived viral DNA in the sample.

In some aspects, the viral nucleic acid sequence includes cell free DNA (cfDNA).

### BRIEF DESCRIPTION OF THE DRAWINGS

FIG. 1 (SEQ ID NO: 16) illustrates a nucleic acid sequence showing MSPCP primers designed to target five CpG sites in the C promoter found in the coordinates 11038-11117 of the reference EBV Type 1 genome NC_007605.1. 72-83 bas base pair amplicon, dark shades indicate extensions for U primers. EBV1: 11044-11118. EBV2: 11029-11102

FIGS. 2A-2E show that MSPCP can determine methylation status and distinguish EBV+ cell lines by latency program. FIG. 2A illustrates a graph that shows methylation index of synthetic oligomer templates that mimic bisulfite-converted DNA methylated and unmethylated Oligo CpG Sequence. FIG. 2B illustrates a graph showing methylation index for A kata, BX1, Rael, AG876, B95.8, LCL, A kata virus, AG876 virus, and B95.8 virus. FIG. 2C illustrates a graph showing methylation index of Rael and B95.8 virus from EBV DNA. FIG. 2D illustrates a graph showing correlation of observed methylation index and expected methylation index. FIG. 2E illustrates a graph showing methylation index of Rael and B95.8 virus with different inputed copy number using MSPCP vs MBD-Cp and MBD-Wp.

FIGS. 3A-3D show that EBV DNA can be detected in plasma from people with HIV and untreated Hodgkin lymphoma. FIG. 3A illustrates a graph that shows EBV and methylated copy numbers in plasma of pretreated HIV positive Classical Hodgkin lymphoma (cHL). FIG. 3B illustrates a graph that shows methylation index of pretreated HIV positive cHL. FIG. 3C illustrates a graph that shows EBV and methylated copy numbers in saliva of JHMI asymptomatic subjects. FIG. 3D illustrates a graph that shows methylation index with MSPCP in in saliva of JHMI asymptomatic subjects.

FIG. 4 (SEQ ID NOs: 4, 17-25) illustrates peaks that show that bisulfite sanger sequencing of Rael and LCL DNA provide orthogonal confirmation of the MSPCP results.

FIG. 5 illustrates a schematic diagram showing gene expression and methylation status and different EBV latency program.

FIG. 6 illustrates a schematic diagram showing sources of EBV DNA in human saliva.

FIG. 7 illustrates a schematic diagram that shows quantitative methylation-specific PCR for EBV Cp.

FIGS. 8A-8B show MSPCP identifies as little as 1% methylation in heterogeneous samples. FIG. 8A illustrates a graph showing that MSPCP accurately classifies EBV+ cell lines by latency pattern based on C promoter methylation. MSPCP is further able to differentiate cell-derived DNA from virion DNA produced by the cognate cell line. Bars represent standard deviation, n=3. FIG. 8B illustrates a graph showing that stocks of Rael (M) and B95.8 Virion (U) EBV DNA were sonicated to ˜200 bp and combined in simulated samples at a copy number of 10,000 EBV copies per reaction. Mixed samples underwent bisulfite conversion and MSPCP. As low as 1% methylation in heterogeneous samples was detected by MSPCP. Bars represent standard deviation, n=3.

FIGS. 9A-9B show characterization of background EBV DNA copy number in an asymptomatic adult population. FIG. 9A illustrates a table that shows demographics of asymptomatic cohort (n=318). All samples are confirmed SARS-COV-2 negative prior to acquisition. FIG. 9B illustrates a graph that shows dot plot of EBV copy number per mL saliva in an asymptomatic population. No relationship between age and EBV presence in saliva was observed.

FIGS. 10A-10B show quantification of EBV DNA methylation in saliva. FIG. 10A illustrates a table showing statistics of EBV Cp methylation. Methylation index was quantified with MSPCP, which was then used to calculate methylated copies per mL of saliva. FIG. 10B illustrates a graph that shows comparison of EBV copy number in saliva and the methylated fraction of these copies using MSPCP. There was no relationship to amount of EBV in the saliva and the size of its methylated fraction. Experiments were performed in technical duplicate, n=82.

FIGS. 11A-11C show that MSPCP specifically amplifies methylated EBV DNA with low minimum input requirement. FIG. 11A illustrates a graph showing that MSPCP quantifies high percent methylation at Cp in Latency I cell lines, but not Latency III cell lines. When analyzing supernatant DNA from cell lines after lytic induction, the supernatant DNA is largely unmethylated. Experiments performed in triplicate via qPCR. FIG. 11B illustrates a graph that shows that MSPCP reproducibly quantifies Cp methylation in as few as 10 starting input copies of EBV DNA. Experiments performed in triplicate via qPCR. FIG. 11C (SEQ ID NOs: 21, 26-30) illustrates peaks showing that bisulfite sanger sequencing of Rael cell DNA orthogonally confirms qPCR results that Cp is densely methylated in Latency I. Arrows represent forward and reverse primer sequence targets. CpG sites targeted by MSPCP are highlighted in red boxes.

FIGS. 12A-12B show that MSPCP is more sensitive than an alternative bead-based method of quantifying EBV DNA methylation. FIG. 12A illustrates a graph that shows that MSPCP was compared to MBD-Biotin bead capture of methylated EBV DNA. MSPCP was more sensitive than MBD beads targeting either the EBV Wp or Cp. FIG. 12B illustrates a graph showing that MSPCP quantifies EBV DNA methylation with high specificity in heterogenous samples. All experiments were performed in triplicate. Bars represent standard deviation of replicates.

FIGS. 13A-13B show that MSPCP quantifies EBV DNA methylation in plasma and saliva. FIG. 13A illustrates graphs showing that plasma cfDNA from pretreatment Hodgkin lymphoma patients have a range of EBV copies/mL of plasma. Despite this, MSPCP measures a high percent methylation at Cp all plasma samples. FIG. 13B illustrates graphs showing that EBV DNA from whole saliva of university staff and students ranges from three to six log 10 copies/mL of saliva. In these samples, MSPCP measures minimal methylation, suggesting it is mostly virion DNA.

### DETAILED DESCRIPTION OF THE DISCLOSURE

The present disclosure is based on the seminal discovery that quantitative methylation-specific PCR for the C Promoter (MSPCP) can sensitively detect, quantify, and differentiate virion-derived and cell/tumor-derived EBV DNA.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

Described herein is a MSPCP, a rapid approach, specifically designed for cfDNA-sized fragments, that can reliably detect as few as 50 methylated copies of EBV DNA. An advantage of MSPCP over previous approaches is the ability to interrogate the methylation status of specific CpG sites, notably those resting under the 3′ end of the primers.

In certain embodiments, the present disclosure provides a method of identifying a viral nucleic acid sequence in a sample as virion DNA or tumor-derived viral DNA including determining a DNA methylation status of the viral nucleic acid sequence, wherein methylation of the viral nucleic acid sequence is indicative of tumor-derived viral DNA and the absence of methylation of the viral nucleic acid sequence is indicative of virion DNA, thereby identifying the viral nucleic acid sequence as virion DNA or tumor-derived viral DNA.

As used herein, the term “nucleic acid” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic can be employed for introduction into, i.e. transfection of, cells, in particular, in the form of RNA which can be prepared by in vitro transcription from a DNA template. The RNA can moreover be modified before application by stabilizing sequences, capping, and polyadenylation.

As used herein the term “viral nucleic acid” refers to the genetic material found within a virus, which can be either deoxyribonucleic acid (DNA) or ribonucleic acid (RNA), and carries the genetic information needed for the virus to replicate inside a host cell. Viruses can have single-stranded or double-stranded DNA or RNA, depending on the virus.

As used herein the term “virion” refers to a complete, infectious virus particle outside a host cell. A virion is the fully assembled structure containing the virus's genetic material (DNA or RNA) encased within a protein coat called a capsid, which allows it to deliver its genome to a host cell to initiate infection. In short it is the “infectious form” of a virus.

As used herein the term “tumor-derived viral DNA” refers to the DNA of a virus that is capable of causing cancer. Tumor-derived viral DNA are from tumor associated viruses, which are viruses that can cause cancer by interfering with cell cycle control and immortalization. DNA of tumor associated viruses may be non-enveloped or enveloped, and their genomes are either circular or double-stranded. The oncogenes of DNA tumor viruses are an integral part of the viral genome and are required for viral replication. Tumor-derived viral DNA, also known as circulating tumor DNA (ctDNA), is viral DNA from tumor cells that may exist in the blood as cell-free DNA. Examples of tumor associated viruses include but are not limited to EBV, adenoviruses, hepatitis B virus, papillomaviruses, polyomavirus, human papillomavirus (HPV), and simian virus 40 (SV40).

In some aspects, the tumor-derived viral DNA is from tumor associated with EBV.

EBV also known as human herpesvirus 4, is a member of the herpes virus family. EBV is one of the most common human viruses in the world and spreads through bodily fluids, primarily saliva. EBV genome is made up of linear double-stranded DNA that is about 172 kilobase pairs (kb) long. EBV has many mapped RNA polymerase II promoters, and the mRNAs from these promoters have been assigned to the latent or early/late productive virus cycles.

EBV C promoter (Cp), Q promoter (Qp), W promoter (Wp), and F promoter (Fp) can be found within the viral genome, with Cp and Wp situated within the BamHI C fragment and Wp repeated multiple times within the BamHI W fragment, respectively; the Qp is a distinct promoter region responsible for driving EBNA1 expression in latency type I and II, while Cp and Wp are primarily active in latency type III, allowing for the expression of multiple EBNA proteins depending on the promoter used during transcription. Specific sequences vary depending on the EBV strain, but nucleic acid sequences are accessible through databases such as NCBI GenBank.

In some aspects, the viral nucleic acid sequence is an EBV nucleic acid sequence.

In some aspects, the EBV nucleic acid sequence includes an EBV Cp, an EBV Wp, EBV Qp, or an EBV Fp.

In some aspects, EBV nucleic acid sequence includes an EBV Cp.

In some aspects, determining the DNA methylation status of the viral nucleic acid sequence includes determining the methylation status of EBV Cp.

As sued herein the term “methylation status” refers to a measure of the methylation of DNA, which can be used as a biomarker for various conditions. Epigenetic modifications are linked to various diseases, such as cancer, neurological disorders, and cardiovascular diseases. A common type of epigenetic modification is DNA methylation. DNA is typically methylated at cytosine residues in CpG. Altered methylation profile are implicated in early disease stages, progression and treatment response. Abnormal DNA methylation patterns are associated with many diseases, including but not limited to Alzheimer's disease, schizophrenia, depression, ADHD, Rett syndrome, and Fragile X syndrome, cancer, Hyperglycemia, hyperlipidemia, autoimmune disorders, Prader-Willi syndrome, Angelman syndrome, and immunodeficiency-centromeric instability-facial anomalies syndrome (ICF) syndrome. EBV-associated diseases are characterized by aberrant methylation of the EBV DNA and the host genome. EBV gene methylation helps the virus evade the immune system and persist in host cells.

The methylation status of the EBV Cp is important for regulating the expression of EBV genes and the virus's lifecycle. The methylation status of the EBV Cp varies during different lytic cycles. In most infected cells, the EBV genome is epigenetically suppressed, resulting in low EBV protein expression. The methylation status of the EBV Cp varies depending on the context. In tumors, the Cp is methylated, silent, and in more restricted forms of latency. For example, in nasopharyngeal carcinoma (NPC), the EBV C promoter is predominantly methylated. In latency II tumors, the LMP1 promoter is unmethylated and active. In oral hairy leukoplakia, the EBER promoters are constitutively active and hypomethylated, except in patients with AIDS.

DNA methylation status can be assessed using various techniques and assays including but not limited to PCR amplification, quantitative PCR (qPCR), bisulfite conversion, bisulfite sequencing, pyrosequencing, bead array, cytosine extension assay (CEA), methylated DNA immunoprecipitation (MeDIP), whole genome bisulfite sequencing (WGBS), reduced representation bisulfite sequencing (RRBS), methyl-MiniSeq, methyl-sensitive cut counting (MSCC), liquid chromatograph-mass spectrometry (LC-MS), methylation sensitive restriction enzyme (MSRE) digestion, and affinity enrichment, Differential enzymatic cleavage of DNA, microarray, next-generation sequencing (NGS), sanger sequencing, and mass spectrometry.

In some aspects, determining the DNA methylation status of EBV Cp includes performing a methylation specific PCR.

In some aspects, performing the methylation specific PCR includes performing a qPCR.

As used herein the term “methylation specific PCR” (MSP) refers to a technique used to analyze DNA methylation patterns in CpG islands using polymerase chain reaction (PCR). PCR is a laboratory technique that amplifies specific segments of DNA or RNA for analysis. This PCR is performed using two primer pairs to detect methylated and unmethylated DNA.

In some aspects, performing the methylation specific PCR includes using primers targeting CpG islands in EBV Cp.

In some aspects, the CpG islands are located within coordinates 11038-11117 of EBV Type 1 reference genome NC_007605.1.

There are two main strains of EBV: type 1 and type 2. Type 1 is more prevalent and has a greater ability to immortalize cells. The EBNA2 and EBNA3 genes are the only genes that can distinguish between type 1 and type 2 EBV strains. EBV reference genome NC_007605.1 is used to compare EBV sequences and identify methylation status. Primers for the PCR may be designed to target five CpG sites in the Cp found in the coordinates 11038-11117 of the reference EBV Type 1 genome NC_007605.1. Disclosed herein is an MSP that utilizes methylated-CpG targeting (M) primer sequences: forward: 5′-CGTTTTATTTGGGAGGAGCGAC-3′ (SEQ. ID NO. 4); reverse: 5′-ACCACTATACTTTACGAACCCTACG-3′ (SEQ. ID NO. 5) and unmethylated-CpG targeting (U) primers: forward: 5′-GAATAATGTTTTATTTGGGAGGAGTGAT-3′ (SEQ. ID NO. 6); reverse: 5′-AAACCACTATACTTTACAAACCCTACA-3′ (SEQ. ID NO. 7).

In some aspects, the primers include: a) methylated-CpG targeting (M) primers and b) unmethylated-CpG targeting (U) primers.

In some aspects, the M primers include a nucleic acid sequence including SEQ ID NO:4 and SEQ ID NO:5.

In some aspects, the U primers include a nucleic acid sequence including SEQ ID NO:6 and SEQ ID NO:7.

In one aspect, a methylated status of the viral DNA is indicative of presence of tumor-derived viral DNA and/or tumor-derived viral DNA in the sample.

Virion DNA is generally considered to be unmethylated, as most viruses do not methylate their own DNA within the virion particle. However, once the viral DNA enters a host cell, it can become methylated depending on the host cell's mechanisms. In some cases, like with EBV, the viral DNA can become methylated when it is integrated into the host cell during a latent infection phase. Described herein is the finding that EBV DNA detected in the saliva of an unselected general population is unmethylated, therefor readily distinguishable from the EBV tumor DNA extracted from biopsy material and from EBV DNA detected in plasma from people with cancer, which is highly methylated.

In another aspect, an unmethylated status of the viral DNA is indicative of absence of tumor-derived viral DNA and/or tumor-derived viral DNA in the sample.

“EBV copy number” refers to the number of copies of the EBV genome in a sample. The copy number can be measured in a sample and used to predict the development and progression of EBV-associated diseases. EBV copy number can vary depending on the disease state and type of tissue or fluid sample being analyzed. The EBV copy number can be measured using a quantitative PCR assay that amplifies the large internal repeats of the EBV genome. EBV has relatively stable copy number of EBV thought to be the result of a balance between the host and virus factors. In a nonlimiting manner the sample may contain more than 1000 EBV copy number. In a nonlimiting manner the sample may contain about 10 to 1000 EBV copy number. The sample may contain less than about 10, 20, 30, 40, 50, 60, 70, 80, 90, 100, 200, 300, 400, 500, 600, 700, 800, 900, or 1000 EBV copy number.

In some aspects, the sample includes less than about 300 EBV copy number.

In some aspects, the sample includes less than about 100 EBV copy number.

In some aspects, the sample includes about 50 EBV copy number.

As used herein, a “sample” or “biological sample” is meant to refer to any “biological specimen” collected from a subject, and that is representative of the content or composition of the source of the sample, considered in its entirety. A sample can be collected and processed directly for analysis or be stored under proper storage conditions to maintain sample quality until analyses are completed. Ideally, a stored sample remains equivalent to a freshly collected specimen. The source of the sample can be an internal organ, vein, artery, or even a fluid. Non-limiting examples of sample include blood, plasma, urine, saliva, sweat, organ biopsy, cerebrospinal fluid (CSF), tear, semen, vaginal fluid, feces, skin, breast milk, and hair.

In some aspects, the sample is a tissue biopsy, plasma, saliva, tumor sample and/or blood sample.

In one aspect, the sample is a plasma sample.

In another aspect, the sample is a whole saliva sample.

In certain embodiments, the present disclosure provides a method of diagnosing cancer in a subject comprising identifying a viral nucleic acid sequence as tumor-derived viral DNA in a sample from the subject, wherein identifying the viral nucleic acid sequence as tumor-derived viral DNA includes determining a DNA methylation status of the viral nucleic acid sequence, wherein detecting methylated tumor-derived viral DNA is indicative of presence of cancer is the subject, thereby diagnosing the subject with cancer.

The term “subject” as used herein refers to any individual or patient to which the subject methods are performed. Generally, the subject is human, although as will be appreciated by those in the art, the subject may be a non-human animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “cancer” refers to a group diseases characterized by abnormal and uncontrolled cell proliferation starting at one site (primary site) with the potential to invade and to spread to other sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof.

Examples of cancers include but are not limited to Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Childhood; Brain Tumor, Glioblastoma, Adult; Brain Tumor, Glioblastoma, Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood’, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (OsteosarcomaVMalignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In some aspects, the cancer is an EBV-related cancer.

As used herein the term “EBV-related cancer” refers to a cancer that is linked to the EBV. EBV can cause genetic changes in cells that make them more likely to become cancerous, thereby increasing the risk of developing certain cancers. Examples of EBV-related cancers include but are not limited to nasopharyngeal cancer, Burkitt lymphoma, Hodgkin lymphoma, diffuse large B cell lymphoma, stomach (gastric) cancer, immunoblastic lymphoma, lymphoepithelioma-like carcinoma (LELC), NK/T cell lymphoma, aggressive NK-cell leukemia.

In some aspects, the EBV-related cancer is a blood cancer or an epithelial cancer.

As used herein the term “blood cancer” refers to a cancer that affects blood, bone marrow, or the lymphatic system. It occurs when blood cells develop abnormally, often due to DNA mutations. Some common types of blood cancer include but are not limited to leukemia, lymphoma, and myeloma.

As used herein the term “epithelial cancer” refers to a type of cancer that forms in epithelial tissue, which lines most organs, internal passageways, and skin. Examples of epithelial cancers include but are not limited to adenocarcinoma, basal cell carcinoma, gastric carcinoma, nasopharyngeal carcinoma, Squamous cell carcinoma, Renal cell carcinoma (RCC), hepatocellular carcinoma (HCC), acinar cell carcinoma, adnoid cystic carcinoma, adrenal cortex carcinoma, ampulla of vater adenocarcinoma, anus adenocarcinoma, appendix adenocarcinoma, breast lobular carcinoma, cervical adenocarcinoma, cholangiocarcinoma, and choriocarcinoma.

In some aspects, the blood cancer is Burkitt lymphoma, diffuse large B cell lymphoma, Hodgkin lymphoma, or NK/T cell lymphoma.

Burkitt lymphoma is a cancer of the lymphatic system, particularly B lymphocytes found in the germinal center. It is an aggressive non-Hodgkin B-cell lymphoma. The disease is associated with EBV, human immunodeficiency virus (HIV), and chromosomal translocations that cause the overexpression of oncogene c-myc.

Diffuse large B-cell lymphoma is a cancer of B cells, a type of lymphocyte that is responsible for producing antibodies. It is the most common form of non-Hodgkin lymphoma among adults. Diffuse Large B-Cell Lymphoma is an aggressive type of non-Hodgkin lymphoma that can arise in lymph nodes or outside of the lymphatic system.

Hodgkin lymphoma is a type of blood cancer that occurs when malignant cells form in the lymph system.

In some aspects, the epithelial cancer is gastric carcinoma, or nasopharyngeal carcinoma.

Gastric carcinoma, also known as stomach cancer, is a cancer that develops in the stomach's lining. It is the fifth most common cancer worldwide and the third leading cause of cancer deaths.

Nasopharyngeal carcinoma is cancer that happens in the nasopharynx, which sits behind the nose and above the back of the throat. It is the most common cancer originating in the nasopharynx, most commonly in the postero-lateral nasopharynx or pharyngeal recess, accounting for 50% of cases.

In some aspects, the viral nucleic acid sequence is an EBV nucleic acid sequence.

In some aspects, the EBV nucleic acid sequence includes an EBV C promoter (Cp), an EBV W promoter (Wp), EBV Q promoter (Qp), or an EBV F promoter (Fp).

In some aspects, the EBV nucleic acid sequence includes an EBV Cp.

In some aspects, determining the DNA methylation status of the viral nucleic acid sequence comprises determining the methylation status of EBV Cp.

In some aspects, determining the DNA methylation status of the viral nucleic acid sequence comprises determining the methylation status of EBV Cp and/or EBV Wp.

In some aspects, the DNA methylation status is indicative of an EBV latency program.

In some aspects, the EBV latency program is type I latency program, type II latency program, or type III latency program.

In some aspects, determining the DNA methylation status of the viral nucleic acid sequence includes performing a methylation specific PCR.

In some aspects, performing the methylation specific PCR comprises performing a qPCR.

In some aspects, performing the methylation specific PCR includes using primers targeting CpG islands in EBV Cp.

In some aspects, the CpG islands are located within coordinates 11038-11117 of EBV Type 1 reference genome NC_007605.1.

In some aspects, the primers include: a) methylated-CpG targeting (M) primers and b) unmethylated-CpG targeting (U) primers.

In some aspects, the M primers comprise a nucleic acid sequence including SEQ ID NO:4 and SEQ ID NO:5.

In some aspects, the U primers comprise a nucleic acid sequence including SEQ ID NO:6 and SEQ ID NO:7.

In one aspect, a methylated status of the viral DNA is indicative of presence of tumor-derived viral DNA in the sample.

In another aspect, an unmethylated status of the viral DNA is indicative of absence of tumor-derived viral DNA in the sample.

In some aspects, the sample includes less than about 300 EBV copy number.

In some aspects, the sample includes less than about 100 EBV copy number.

In some aspects, the sample includes about 50 EBV copy number.

In some aspects, the viral nucleic acid sequence includes cell free DNA (cfDNA).

In some aspects, the sample is a biopsy tissue, plasma, saliva, tumor, or blood sample.

In one aspect, the sample is a plasma sample.

In another aspect, the sample is a whole saliva sample.

In some aspects, the subject is a mammal.

In some aspects, the mammal is human.

In certain embodiments, the present disclosure provides a kit including: a) methylated-CpG targeting (M) primers, wherein the M primers include a nucleic acid sequence comprising SEQ ID NO:4 and SEQ ID NO:5, b) unmethylated-CpG targeting (U) primers, wherein the U primers include a nucleic acid sequence comprising SEQ ID NO:6 and SEQ ID NO:7, and c) instructions for using the U and M primers for methylation specific PCR on a biological sample.

In further aspects, the kit includes a sample collection device for obtaining the biological sample from a subject.

In some aspects, the presence of methylation in the sample after PCR is indicative of an EBV-related cancer.

In some aspects, the sample is a biopsy tissue, plasma, saliva, tumor or blood sample.

In one aspect, the sample is a plasma sample.

In another aspect, the sample is a whole saliva sample.

In some aspects, the sample includes viral nucleic acid sequences.

In some aspects, the viral nucleic acid sequences include cfDNA or cell-associated DNA.

In some aspects, the EBV-related cancer is a blood cancer or an epithelial cancer.

In some aspects, the blood cancer is Burkitt lymphoma, diffuse large B cell lymphoma, Hodgkin lymphoma, or NK/T cell lymphoma.

In some aspects, the epithelial cancer is gastric carcinoma, or nasopharyngeal carcinoma.

In some aspects, the subject is a mammal.

In some aspects, the mammal is human.

In certain embodiments, the present disclosure provides a method of treating cancer in a subject in need thereof including: a) identifying a viral nucleic acid sequence as tumor-derived viral DNA in a sample from the subject, wherein identifying the viral nucleic acid sequence as tumor-derived viral DNA includes determining a DNA methylation status of the viral nucleic acid sequence, wherein detecting methylated tumor-derived viral DNA is indicative of presence of cancer is the subject and wherein an unmethylated status of the viral DNA is indicative of absence of tumor-derived viral DNA in the sample; and b) administering an anti-cancer treatment to the subject diagnosed with cancer, thereby treating the cancer in the subject.

The term “treatment” is used interchangeably herein with the term “therapeutic method” or “therapy” and refers to 1) therapeutic treatments or measures that cure, slow down, lessen symptoms of, and/or halt progression of a diagnosed pathologic conditions or disorder, and/or 2) prophylactic/preventative measures. Those in need of treatment may include individuals already having a particular medical disorder as well as those who may ultimately acquire the disorder (i.e., those needing preventive measures).

The terms “therapeutically effective amount”, “effective dose,” “therapeutically effective dose”, “effective amount,” or the like refer to that amount of the subject agent that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of the disease). Such amount should be sufficient to eliminate tumor cells. The effective amount can be determined as described herein.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to inhalation, otic, buccal, conjunctival, dental, endocervical, endosinusial, endotracheal, enteral, epidural, extra-amniotic, extracorporeal, hemodialysis, in-filtration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intra-tumor, intratympanic, intrauterine, intravascular, intravenous, intravenous bolus, intravenous drip, intravesical, intravitreal, intracapsular, intraorbital, intracutaneous, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic administration subcuticular, intraarticulare, subcapsular, intrasternal, ocular administrations, as well infusion, and nebulization, or any combination thereof.

As used herein the term “anti-cancer treatment” refers to any treatment that prevents or stops, or slows, the growth of cancer. Examples of anti-cancer treatment include but are not limited to chemotherapy, radiation therapy, immunotherapy, resection surgery, hormone therapy, targeted therapy, and stem cell or bone marrow transplant.

In some aspects, the anti-cancer treatment is chemotherapy, radiation therapy, immunotherapy, resection surgery, hormone therapy, stem cell or bone marrow transplant, or a combination thereof.

Chemotherapy, also known as chemo, is a cancer treatment that uses drugs to kill or stop the growth of cancer cells. Chemotherapy drugs work by disrupting the cell cycle. Some drugs stop DNA from copying itself, which prevents cancer cells from multiplying.

Radiation therapy is a cancer treatment that uses high doses of radiation to kill cancer cells and shrink tumors. It is also known as irradiation or radiotherapy.

Immunotherapy is a treatment that uses substances to activate or suppress the immune system to help fight disease. Immunotherapies can be used to treat a variety of conditions, including cancer, infections, and other diseases. Immunotherapy can target and destroy proteins or receptors on cancer cells to prevent them from evading the immune system. Examples of immunotherapies include but are not limited to immune checkpoint inhibitors, T-cell transfer therapy, and antibodies.

Resection surgery is a surgical procedure that removes tissue, part, or all of an organ. It can be performed for a variety of reasons, including to remove diseased or cancerous tissue, or to treat or cure a disease process.

Hormone therapy is a treatment that involves adding, blocking, or removing hormones to treat a variety of conditions including cancer. It is a cancer treatment that slows or stops the growth of cancer that uses hormones to grow. Hormone therapy is also called hormonal therapy, hormone treatment, or endocrine therapy.

As used herein the term “stem cell transplant” or “bone marrow transplant” refers to the process of replacing damaged blood cells with healthy stem cells obtained from bone marrow, meaning bone marrow transplant is just one type of stem cell transplant where the stem cells are specifically harvested from the bone marrow. Both are used to treat conditions like leukemia, lymphoma, and certain blood disorders by restoring healthy blood cell production.

In some aspects, the viral nucleic acid sequence is an EBV nucleic acid sequence.

In some aspects, the EBV nucleic acid sequence includes an EBV C promoter (Cp), an EBV W promoter (Wp), EBV Q promoter (Qp), or an EBV F promoter (Fp).

In some aspects, EBV nucleic acid sequence includes an EBV Cp.

In some aspects, determining the DNA methylation status of the viral nucleic acid sequence includes determining the methylation status of EBV Cp.

In some aspects, determining the DNA methylation status of the viral nucleic acid sequence includes determining the methylation status of EBV Cp and/or EBV Wp.

In some aspects, determining the DNA methylation status of the viral nucleic acid sequence includes performing a methylation specific PCR.

In some aspects, performing the methylation specific PCR includes performing a qPCR.

In some aspects, performing the methylation specific PCR includes using primers targeting CpG islands in EBC Cp.

In some aspects, the CpG islands are located within coordinates 11038-11117 of EBV Type 1 reference genome NC_007605.1.

In some aspects, the primers include: a) methylated-CpG targeting (M) primers and b) unmethylated-CpG targeting (U) primers.

In some aspects, the M primers include a nucleic acid sequence comprising SEQ ID NO:4 and SEQ ID NO:5.

In some aspects, the U primers include a nucleic acid sequence comprising SEQ ID NO:6 and SEQ ID NO:7.

In some aspects, a methylated status of the viral DNA is indicative of presence of tumor-derived viral DNA in the sample.

In some aspects, the sample includes less than about 300 EBV copy number.

In some aspects, the sample includes less than about 100 EBV copy number.

In some aspects, the sample includes about 50 EBV copy number.

In some aspects, the viral nucleic acid sequence includes cell free DNA (cfDNA).

In some aspects, the cancer is an EBV-related cancer.

In some aspects, the EBV-related cancer is a blood cancer or an epithelial cancer.

In some aspects, the blood cancer is Burkitt lymphoma, diffuse large B cell lymphoma, Hodgkin lymphoma, or NK/T cell lymphoma.

In some aspects, the epithelial cancer is gastric carcinoma, or nasopharyngeal carcinoma. In some aspects, the sample is a biopsy tissue, plasma, saliva, tumor or blood sample.

In one aspect, the sample is a plasma sample.

In another aspect, the sample is a whole saliva sample.

In some aspects, the subject is a mammal.

In some aspects, the mammal is human.

Presented below are examples discussing methods of identifying tumor-derived viral DNA based on methylation status contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Experimental Methods

### Cell Culture and DNA Isolation

Akata, B95.8, LCL, Namalwa, Rael, and Raji cells were grown in complete RPMI media (RPMI, 10% FBS, 1% penicillin-streptomycin, 1% L-glutamine). BX1 Akata cells were grown in complete RPMI media supplemented with 1% geneticin. Cultures were maintained in 37° C. incubators with 5% CO₂.

Lytic induction of Akata and BX1 Akata cells was done by culturing the cells in complete RPMI supplemented with anti-human IgG at a concentration of 10 μg/mL for four days. Lytic induction of B95.8, Rael, and AG876 cells was done by culturing cells in complete RPMI supplemented with TPA at 20 ng/ml and sodium butyrate at 3 mM for four days. Following stimulation, cells were pelleted, and supernatant media was filtered through a Millex 0.45 μm filter. Filtered media was concentrated to 1 mL using Amicon Ultra-15 Centrifugal Filter Unit tubes.

If isolating virion DNA from cell supernatant, to remove contaminating nuclear EBV DNA from viral isolates, concentrated virion media was treated with DNase I for 10 mins at 37° C., per manufacturer protocol. EDTA was added for a final concentration of 5 mM and mixed by pipette, then samples immediately underwent isolation.

Cell and virion DNA was extracted with the DNeasy Blood and Tissue Kit per manufacturer instructions and eluted in EB buffer. DNA concentrations were determined by Qubit High Sensitivity reagents. EBV copy number was quantified by qPCR with a Namalwa cell DNA standard curve using TaqMan Universal Master Mix II, custom primers and probes targeting the EBV BamW region.

### Human Sample Selection and Isolation

Tumors were selected after EBV DNA was detected by Illumina sequencing of tumor tissue. Tumors were confirmed EBV(+) using EBER in situ hybridization by a pathologist. DNA was isolated from FFPE tumor samples as previously described.

Cell-free DNA was isolated from plasma specimens as previously described.

Saliva samples were collected by the Johns Hopkins Stand-alone Covid Lab. Donors were undergraduate and graduate students of Johns Hopkins, as well as university and hospital staff. Approximately 5 mL of saliva was received into 50 mL Falcon tubes containing no preservatives. Samples were processed, described as follows, for total nucleic acid (TNA) on the day of acquisition (<12 hrs). TNA was extracted using the MagMAX Viral/Pathogen II kit using Opentrons OT2 and Kingfisher Flex robots. TNA was eluted at a volume of 50 μl; following Covid-testing, 40 μl remained for our experiments. Only Covid-negative samples were used for these experiments. From TNA eluate, EBV copy number was quantified by qPCR using TaqMan Universal Master Mix II, custom primers (forward: 5′-CCCAACACTCCACACC-3′ (SEQ. ID NO. 1); reverse: 5′-TCTTAGGAGCTGTCCGAGGG-3′ (SEQ. ID NO. 2)) and probe (5′-/56-FAM/CACACACTACACACACCCACCCGTCTC/3BHQ_1/-3′ (SEQ. ID NO. 3)) targeting the EBV BamW region.

### Primer Design and Synthetic Templates

M13 tags are indicated in bold, underlined, and italicized.

BamW_Native-forward:
SEQ ID NO: 1
CCCAACACTCCACACC
BamW_Native reverse:
SEQ ID NO: 2
TCTTAGGAGCTGTCCGAGGG
Probe:
SEQ ID NO: 3
FAM/CACACACTACACACACCCACCCGTCTC/3BHQ_1/-3
MSPCP_M-forward:
SEQ ID NO: 4
CGTTTTATTTGGGAGGAGCGAC
MSPCP_M-reverse:
SEQ ID NO: 5
ACCACTATACTTTACGAACCCTACG

U Primers are extended in underlined regions at 5′ end to match Tm of M primers.

MSPCP_U-forward:
SEQ ID NO: 6
GAATAATGTTTTATTTGGGAGGAGTGAT
MSPCP_U-reverse:
SEQ ID NO: 7
AAACCACTATACTTTACAAACCCTACA
Cp_Native_M13-forward:
SEQ ID NO: 8
TGTAAAACGACGGCCAGTGTTGAGAGGTTAGTGTTTTAAATATGTAT
TTTAGG
Cp_Native_M13-reverse:
SEQ ID NO: 9
CAGGAAACAGCTATGACCTAAAACCCCTTTACCCAACCC

Synthetic template for MMM-MM. Primer sites are underlined. Target CG sites are in bold.

MSPCP_MMM-MM Gblock-forward:
SEQ ID NO: 10
ACCTTGTTGGCGGGAGAAGGAATAACGTTTTATTTGGGAGGAGCGACG
GATTATAGCCAATAAGAGAGCTCAAGACGTAGGGTTCGTAAAGTATAG
TGGTTTCGTGGGACCTTAGAGGTGGAGCA

Synthetic template for UUU-UU. Primer sites are underlined. Target CG sites are in bold.

MSPCP_MMM-MM Gblock-forward:
SEQ ID NO: 11
ACCTTGTTGGCGGGAGAAGGAATAATGTTTTATTTGGGAGGAGTGATG
GATTATAGCCAATAAGAGAGCTCAAGATGTAGGGTTTGTAAAGTATAG
TGGTTTCGTGGGACCTTAGAGGTGGAGCA
MSPCP_Native Quant-forward:
SEQ ID NO: 12
GGAGAAGGAATAACGCCTTATCT
MSPCP_Native_Quant-reverse:
SEQ ID NO: 13
CCACTATACTTTGCGAGCCC
Cp_Bisulfite_M13-forward:
SEQ ID NO: 14
TGTAAAACGACGGCCAGTTGTTATAAGATTATTAAGTTGGTGTAAA
Cp_Bisulfite_M13-reverse:
SEQ ID NO: 15
CAGGAAACAGCTATGACCTTTACAACAAAACACAAAATTTTTATAA

Bisulfite-specific primers for Cp were designed with Primer Suite using the EBV Type 1 genome from NCBI (accession number NC_007605.1). Tm prediction of candidate primers was carried out using the OligoAnalyzer tool available from IDT. Potential primer dimer identification was done using both OligoAnalyzer and PrimerDimer. Candidate primers with a dimerization AG of <−9.00 kcal/mole were excluded. The methylated-CpG targeting (M) primer sequences are: forward: 5′-CGttttatttgggaggagCGaC-3′ (SEQ. ID NO. 4); reverse: 5′-accactatactttaCGaaccctaCG-3′ (SEQ. ID NO. 5). The unmethylated-CpG targeting (U) primers are: forward: 5′-gaataaTGttttatttgggaggagTGaT-3′ (SEQ. ID NO. 6); reverse: 5′-aaaccactatactttaCAaaccctaCA-3′ (SEQ. ID NO. 7). U primers are slightly extended to ensure M and U primers have similar Tm. The following primers, containing an M13 tag on the 5′ end, were used for bisulfite for Cp:

forward:
(SEQ. ID NO. 8)
5′-TGTAAAACGACGGCCAGTGTTGAGAGGTTAGTGTTTTAAAATATA
GTATTTTAGGAATAAGG-3′;
reverse:
(SEQ. ID NO. 9)
5′-CAGGAAACAGCTATGACCtaaaacccctttacccaaccc-3′.

An optimal primer annealing temperature was determined using a PCR temperature gradient ranging from 55° C. to 65° C., conducting qPCR on template DNA and no-template controls, performed in technical duplicate.

Synthetic MSPCP template sequences were generated using bases 11000-11150 from EBV Type 1 genome NC_007605.1. Primer binding site non-CpG sequences were converted to correspond with bisulfite-converted sequence. CpGs in primer binding sites were changed to converted sequences in U-designated oligomers. Synthetic MSPCP templates were generated as gBlock Gene Fragments (IDT). The Gblock sequences were as follows:

```
  (SEQ. ID NO. 10)
M = 5′-ACCTTGTTGGCGGGAGAAGGAATAACGTTTTATTTGGGAG
GAGCGACGGATTATAGCCAATAAGAGAGCTCAAGACGTAGGGTTCGT
AAAGTATAGTGGTTTCGTGGGACCTTAGAGGTGGAGCA-3′;
  (SEQ. ID NO. 11)
U = 5′-ACCTTGTTGGCGGGAGAAGGAATAATGTTTTATTTGGGAG
GAGTGATGGATTATAGCCAATAAGAGAGCTCAAGATGTAGGGTTTGT
AAAGTATAGTGGTTTCGTGGGACCTTAGAGGTGGAGCA-3′.
```

Oligomers were resuspended per manufacturer recommendations and concentration was confirmed using Tapestation High Sensitivity D1000 Tape and Reagents. Copy number per microliter was derived based on the molecular weight and concentration of the oligomers, and templates were serially diluted to desired copy number in water. Oligomers used for qPCR standards were aliquoted in desired copy number and stored in TE buffer.

### Quantitative Methylation Analysis

Bisulfite conversion was carried out using EZ DNA Methylation Lightning Kit according to manufacturer instructions. Briefly, up to 20 μL of template was mixed with 130 μL of Conversion Reagent. Samples were incubated in a thermocycler for: 95° C. for 15 minutes, 55° C. for 1 hour, and 4° C. hold. Conversion was completed using the provided column and desulphonation reagents and purified. Converted DNA was eluted at 21 μl in EB Buffer.

qPCR was performed using Power SYBR Master Mix at a reaction volume of 25 μl. Primers at a final concentration of 250 nM were used in parallel for all samples, with each reaction containing 5 μl of converted template. The synthetic MSPCP templates described above were used as standards for copy number quantification. A methylation control (Rael DNA), an unmethylated control (B95.8 virion DNA), and a no-template control were included in each experiment to confirm complete bisulfite conversion and primer specificity. All samples and controls were run in technical duplicate. Standard curve amplification was analyzed between runs to monitor for standard degradation. Using a Bio-Rad CFX96 PCR Detection System, qPCR was performed with the following settings: 95° C. for 10 mins; 40 cycles of 95° C. for 30 secs, 60° C. for 60 secs; melt curve analysis by instrument standard protocol.

### Bisulfite Sanger Sequencing

Regions of interest were amplified using custom PCR primers described above. PCR amplicons were purified with AMPure XP Beads and eluted in EB buffer. Single amplicon purity was confirmed with agarose gel electrophoresis, visualized with GelGreen. Amplicons were submitted to the Johns Hopkins University Genetic Resources Core Facility for Sanger sequencing of forward and reverse reads according to core facility protocol.

### MethylMiner MBD Assay

MethylMiner Methylated DNA Enrichment Kit assay was performed as previously described. Briefly, streptavidin Dynabeads were washed twice with 1× wash buffer and then incubated with MBD-biotin for 1 hour at RT on an end-over-end mixer. Coupled beads were washed three times using 1× wash buffer. Sample gDNA was sonicated to 600 bp. Sample DNA was loaded at 1 μg per reaction; samples with low concentration were supplemented with sonicated K562 DNA to achieve this DNA input. Samples were incubated with MBD-coupled beads for 1 hours at RT on an end-over-end mixer. Beads were incubated on a magnet rack for 1 minute, then supernatant was collected as no-capture DNA. Beads were washed once more with 1× wash buffer, and this supernatant was collected as no-capture. Beads were then washed twice each with 300 mM, 450 mM, and 2000 mM sodium elution buffer, sequentially. Elutions were collected from each wash. Eluted DNA in each fraction was quantified by qPCR in technical duplicate. Quantification for BamW was done with TaqMan, custom primers, custom probe, as described above, and a Namalwa standard curve. Quantification for native Cp was done with Power SYBR qPCR master mix, custom primers for Cp (forward: 5′-ggagaaggaataaCGccttatct-3′ (SEQ. ID NO. 12); reverse: 5′-ccactatactttgCGagccc-3′ (SEQ. ID NO. 13)), and a Namalwa standard curve.

### Data Analysis

qPCR data was analyzed using CFX Maestro software. Sanger sequencing was analyzed using 4peaks. Percent methylation was calculated as percentages of absolute copy numbers detected between M and U within the same sample using the formula

$$Percent methylation = \frac{M Copies}{M Copies + U Copies} \times 100$$

Methylated copies/mL of liquid samples was derived using Methylated copy number=Percent methylation×native copy number/mL. Data analysis and plot generation were performed using Rstudio. Correlation was determined by Pearson correlation test.

Although the invention has been described with reference to the presently preferred embodiment, it should be understood that various modifications can be made without departing from the spirit of the invention. Accordingly, the invention is limited only by the following claims.

### Example 2

### MSPCP Primers Preferentially Amplify

### Corresponding Synthetic Targets

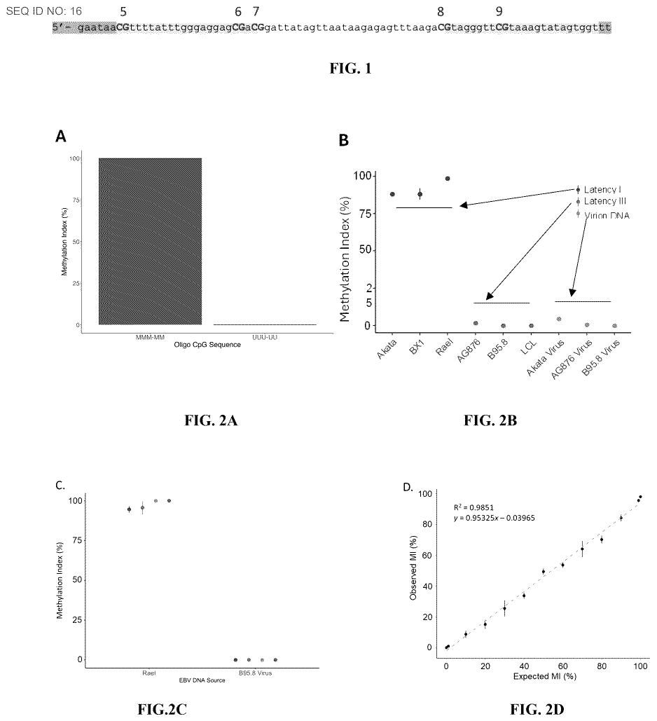

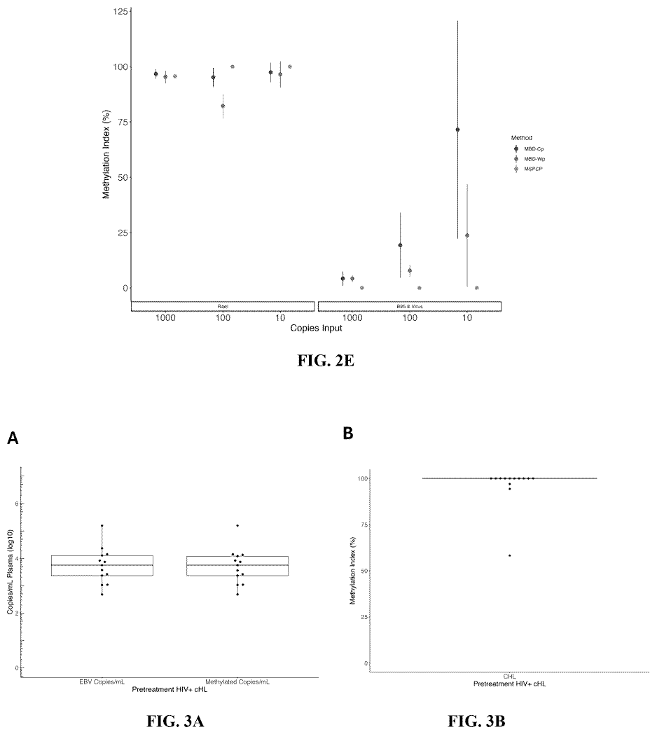

MSPCP primers were designed to target five CpG sites in the C promoter found in the coordinates 11038-11117 of the reference EBV Type 1 genome (NC_007605.1) (FIG. 1). The target region is outlined in FIG. 1. It was first sought to validate MSPCP using synthetic oligomer templates that mimic bisulfite-converted DNA with all methylated (MMM-MM) or unmethylated (UUU-UU) CpG sequences. MSPCP quantification was performed on triplicate reactions of 10,000 copies of template. It was observed that M and U primers only amplified corresponding synthetic oligomer sequences (FIG. 2A). A cross-amplification of approximately 1 in 10,000 copies in pure synthetic oligo template was observed.

### Example 3

### MSPCP Distinguishes Between EBV Latency Patterns

To determine if MSPCP could distinguish EBV+ cell lines by latency program, Cp methylation in a variety of cell lines were assessed, as well as their cognate virions (FIG. 2B). EBV+BL tumor-derived cell lines with Latency I phenotype: Akata, BX1 (a modified Akata cell line with a GFP transgene), and Rael were investigated. All were densely methylated at Cp. In contrast, two lymphoblastoid cell lines (B95.8 and LCLs) are unmethylated at Cp. Many BL cell lines have drifted to a Latency III pattern during culture. AG876 is one such line; qMSP measured no CpG methylation at Cp. Virions from Akata, AG876, and B95.8, were also assayed and these samples showed no methylation. Bisulfite Sanger sequencing of Rael and LCL DNA provided orthogonal confirmation of the MSPCP results (FIG. 4). Thus, MSPCP assessment of Cp methylation paralleled the results of Sanger bisulfite sequencing and reports in the literature.

### Example 4

### MSPCP has a Low Minimum DNA Input Requirement

Next the limit of detection of MSPCP were explored in order to determine the minimum numbers of copies required for precise measurement. Serial dilutions of sonicated Rael cell or B95.8 virion DNA was performed to replicate detection of cell-free DNA fragments (FIG. 2C). Described herein is the disclosure that MSPCP could detect as few as 10 EBV copies in the starting material, both for methylated and unmethylated targets.

Additionally, to investigate the precision in heterogenous samples, in which methylated and unmethylated molecules are intermingled, Rael DNA (as shown above, highly methylated at the Cp locus) with virion DNA (B95.8) were mixed at known ratios to achieve a final copy number of 10,000 input copies (FIG. 2D). Described herein is the finding that methylation percentages observed with MSPCP correlated with the expected percent methylation. These results indicated detection of rare methylated molecules amongst a majority of unmethylated copies and can differentiate methylated and unmethylated molecules in heterogenous samples.

### Example 5

### Comparison of MSPCP and MBD

In order to ascertain if MSPCP was an effective alternative for MBD, parallel comparisons of each method's sensitivity and specificity was performed (FIG. 2E). For these investigations Rael cells was used as a source of highly methylated EBV DNA and B95.8 supernatant as a source of unmethylated EBV DNA. MSPCP distinguished M and U DNA at all dilution concentrations (FIG. 2E). In contrast, MBD distinguished M and U DNA at high copy number but had decreasing sensitivity at lower copy number. These data suggest a role of non-specific binding, or binding of methylated CpG sites adjacent to the qPCR target region.

Additionally, to determine if targeting a repetitive region via MBD analysis improved detection compared to a non-repeat region MBD detection of EBV IR1 was explored, a large repetitive region consisting of five to eight repeated cassettes that also contains the W promoter. Described herein is the finding that targeting IR1 with MBD improves limit of detection approximately 10-fold; however, this improvement does not surpass MSPCP. Described herein is the disclosure that MSPCP offers higher sensitivity as a means to quantify EBV cfDNA methylation in low copy-number samples.

### Example 6

### MSPCP Quantifies EBV DNA Methylation in Solid Tumors and Liquid Samples

MSPCP was applied to Formalin-fixed, paraffin-embedded (FFPE) tumor samples from a variety of EBV(+) cancers. Nine EBV(+) lymphoma specimens, two NPC samples, one gastric carcinoma, and one reactive lymphoid proliferation were evaluated. Values for each surgical specimen are in Table 1 and Table 2. Only one tumor, which was attributed to Covid-19-related lymphoid hyperplasia, showed dense Cp methylation.

TABLE 1
Value of Methylation for Surgical Specimen

| | | Percent |
|---|---|---|
| Sample ID | Diagnosis | Methylation |
| R19-704 | HIV(+) cHL | 99.8 |
| R19-708 | HIV(+) cHL | 94.2 |
| R21-468 | HIV(+) cHL | 97.3 |
| R21-466 | HIV(+) cHL | 98.8 |
| MDL-20-9171-SH | EBV(+) Diffuse Large B-Cell | 100 |
| | Lymphoma, Not Otherwise Specified | |
| MDL-19-8477-FR | EBV(+) Gastric Adenocarcinoma | 100 |
| MDL-20-7712-LF | EBV(+) Lymphoid Hyperplasia | 3.4 |
| MDL-19-8317-CM | Nasopharyngeal Carcinoma | 99.9 |
| MDL-20-8323-PT | Nasopharyngeal Carcinoma | 99.9 |
| MDL-20-5090-JS | Mature T-cell and Natural | 100 |
| | Killer Cell Neoplasm | |

TABLE 2
Formalin-fixed paraffin-embedded EBV(+) tissue
samples analyzed by MSPCP

| | | Percent |
|---|---|---|
| | | Methylation |
| Specimen Category | Diagnosis | (%) |
| EBV(+) Lymphomas | HIV(+) cHL | 99 |
| | HIV(+) cHL | 94 |
| | HIV(+) cHL | 97 |
| | HIV(+) cHL | 98 |
| | Difuse Large B-cell Lymphoma | 100 |
| | NK/T-cel Lymphoma | 100 |
| EBV(+) Carcinomas | Gastric Adenocarcinoma | 100 |
| | Nasopharyngeal Carcinoma | 99 |
| | Nasopharyngeal Carcinoma | 99 |
| EBV(+) Reactive | Immunoblastic Proliferation* | 3.4 |
| Lymphoid Proliferation | | |
| *Reported history of | | |
| SARS-CoV-2 infection | | |

DNA extracted from saliva collected from a university population being screened for Covid-19 were also explored. DNA from 25 EBV(+) saliva specimens were analyzed. In these specimens, there was little to no Cp methylation (max: 1.75%) consistent with the expectation that salivary EBV is almost exclusively virion DNA.

Finally, Cp methylation in plasma cell-free DNA from 13 HIV (+) Hodgkin lymphoma patients were examined prior to cancer treatment. Described herein it the finding that, unlike the saliva samples, the plasma cfDNA was almost entirely methylated at Cp, with only one sample <90% methylated (min: 58.22%).

### Example 7

### Discussion

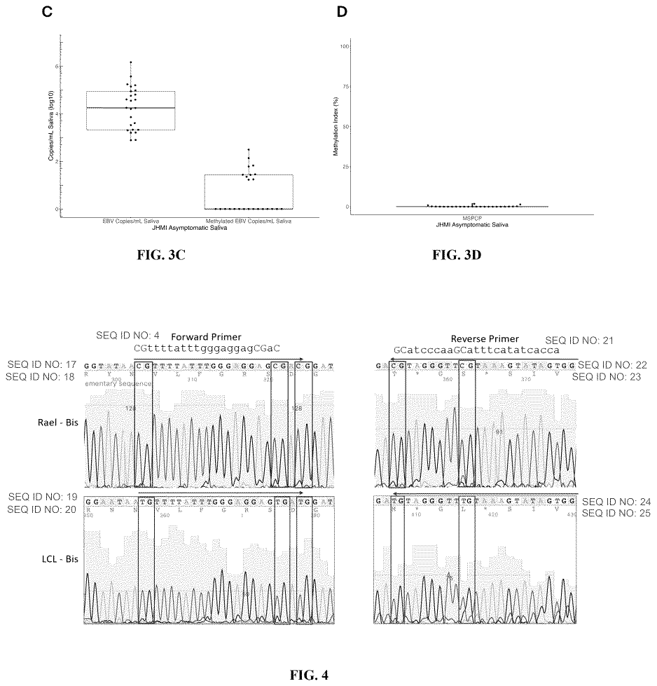

Described herein is the ability of a novel approach, MSPCP, to quantify EBV DNA methylation at the Cp locus. The assay readily distinguishes virion DNA (unmethylated) from viral DNA in tumor cell lines (methylated). Similarly, EBV DNA detected in the saliva of an unselected general population (unmethylated) is readily distinguished from the EBV tumor DNA extracted from biopsy material (highly methylated) and from EBV DNA detected in plasma from people with HIV and untreated Hodgkin lymphoma (highly methylated) (FIGS. 3A-3D). The data presented establishes MSPCP as a sensitive, reproducible technique to characterize CpG methylation at a critical locus in the EBV genome that can be applied to a variety of specimens.

There is growing interest in the characterization of CpG methylation in body fluids, particularly plasma, for detection and monitoring of malignancy. For example, multicancer early detection approaches have demonstrated that ctDNA methylation profiling as developed by GRAIL detects more than 50 cancer types. Others have appreciated the possible application of EBV methylation profiling in clinical specimens for cancer detection—with a narrower focus on populations at especially high risk for EBV malignancy such as southern Chinese men who are at risk for nasopharyngeal carcinoma. Whole viral DNA bisulfite sequencing from plasma is a powerful technique that improves the positive predictive value of detecting EBV DNA in plasma in a prospective screening study. The technique described herein provides detailed analysis for ease of implementation and simplicity insofar as sophisticated sequencing equipment is not required.

Many groups have developed bisulfite-PCR-based assays for detection of methylation in cancer, leveraging this ease of implementation otherwise unavailable to sequencing-based strategies. DREAMing leverages differential melting temperatures in bisulfite-converted DNA to analyze methylation density and epigenetic heterogeneity in NSCLC and myeloid cancers. Multiplex bisulfite PCR can quantify methylation in multiple loci in a single run. Taken a step further, LBx-BCM represents a cartridge-based assay for the automated detection of methylation markers in breast cancer. All of these assays represent the potential for PCR-based methylation quantification in cancer. MSPCP fits into this niche as a means to sensitively quantify methylated EBV DNA in EBV(+) cancers.

A recent report demonstrated the potential for analyzing EBV methylation in saliva to detect nasopharyngeal carcinoma. In targeting a different CpG locus in the C promoter, Zheng et al. demonstrated the high sensitivity achievable through MSP approaches when used in saliva. Their analysis, also informed by bisulfite sequencing, targeted one CpG site, while ours targets five sites under the same primer set. Similarly, Cp methylation is higher in nasal swabs of NPC(+) patients. These results were corroborated using a different locus in two EBV(+) NPC tumors. These studies, together with our results, highlight the potential for EBV methylation analysis in detecting a variety of EBV(+) tumors using solid tissue, plasma, and saliva samples. MSPCP may also offer some prognostic insight by monitoring changes in methylated DNA fractions during treatment.

In a previous investigation, EBV cfDNA methylation was explored as a cancer biomarker using methyl-binding protein-coated beads. This approach has been valuable, but it may be limited in terms of sensitivity and accessibility. Its use is also hampered by a high DNA input requirement in order to obtain robust results, which may be unachievable in low-copy samples. One goal in developing MSPCP was to provide an alternative method for EBV cfDNA methylation assessment, with a focus on faster sample analysis, lower input requirements, and accessibility of reagents. Of note, MSPCP has the advantage of reliable results with limited starting DNA. This feature allows analysis of rare or valuable samples, such as plasma isolates that are used in several different analyses. By using low volumes of sample DNA, more of the original sample can be reserved for more material-intensive experiments, such as bisulfite sequencing.

It is important to consider the scope of information MSPCP can provide. MSPCP is strictly for detection of EBV Cp, as the primers target specific CpGs in the viral promoter.

MSPCP will serve as a valuable tool for both basic research into EBV biology, and clinical applications for detection and monitoring of EBV+ cancers around the globe. It was demonstrated that MSPCP can quantify methylated EBV DNA in tumors, plasma and saliva. The technique can potentially be translated for biomarker detection, particularly in resource-limited settings. Future investigations will expand our understanding of EBV DNA in saliva.

This contrast with the results from saliva samples suggests these EBV DNA molecules are derived from different sources, such as cell turnover in the EBV+ tumor. Furthermore, the high methylation percentage matches the trend seen in the EBV+ tumor analysis. Taken together, these data indicate that MSPCP can sensitively detect and quantify virion-derived and cell/tumor-derived EBV DNA by Cp methylation in human solid and liquid samples.

### Example 8

### Characterizing Saliva Epstein-Barr Virus DNA Methylation in an Asymptomatic Population

Plasma EBV DNA has been used as a cancer biomarker (FIG. 5), but saliva has been ignored due to background EBV DNA in healthy populations. However, saliva can contain biomarkers for distant diseases, such as lung cancer. Described herein is a study that employs a quantitative method, MSPCP, to characterize the EBV DNA landscape in a healthy population.

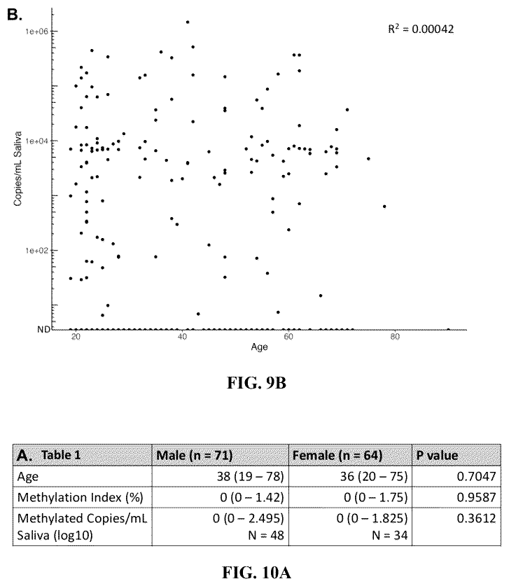

Remnant total nucleic acid isolated from whole saliva (collected from 2021 to 2022) was obtained from 318 asymptomatic university staff/students after SARS-COV-2 negative confirmation. The cohort median age was 33 (range: 19 to 90), with 54% female and 46% male donors. EBV DNA was detected and quantified by qPCR. Methylation was quantified using MSPCP in EBV+ samples (FIGS. 9A-9B).

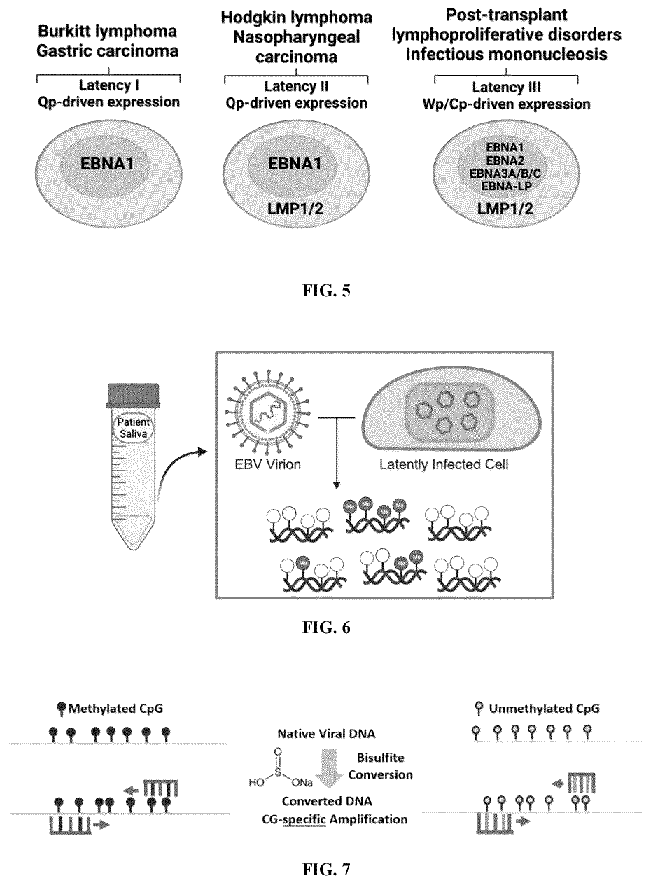

EBV DNA may enter the saliva through several routes. During lytic replication, EBV virions are shed into the saliva. The EBV genomes packaged in these virions are devoid of methylation. EBV-infected oral epithelial cells and oral mucosal lymphocytes may slough into the saliva and contribute to saliva EBV DNA. These viral genomes may have methylation patterns reflective of the infected cells. These sources of EBV DNA create a mosaic of methylated EBV DNA in the saliva (FIG. 6).

EBV+ DNA samples are bisulfite-converted using Zymo Lightning Conversion reagents. Converted DNA is quantified using qPCR with methylation-specific primers that target CpG sites in Cp. Primers target both methylated (M) DNA and unmethylated (U) DNA (FIG. 7).

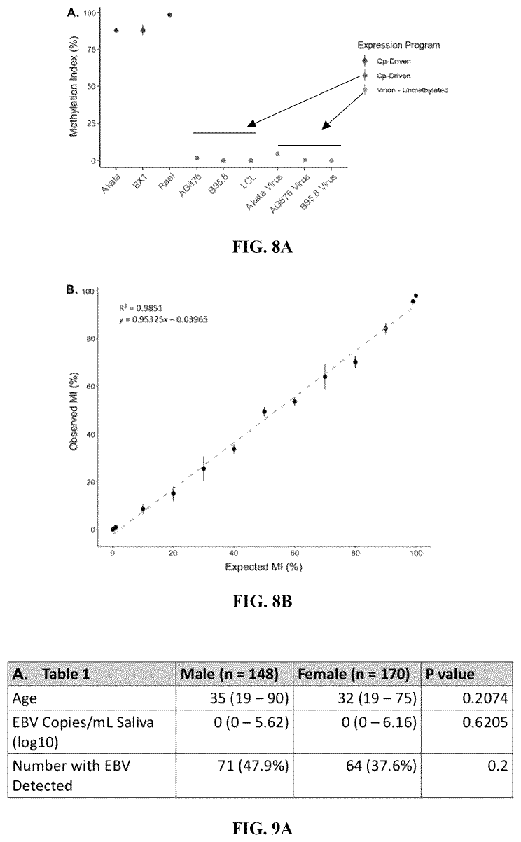

MSPCP accurately classifies EBV+ cell lines by latency pattern based on C promoter methylation. MSPCP is further able to differentiate cell-derived DNA from virion DNA produced by the cognate cell line. Bars represent standard deviation, n=3. Stocks of Rael (M) and B95.8 Virion (U) EBV DNA were sonicated to ˜200 bp and combined in simulated samples at a copy number of 10,000 EBV copies per reaction. Mixed samples underwent bisulfite conversion and MSPCP. As low as 1% methylation in heterogeneous samples was detected by MSPCP (FIGS. 8A-8B).

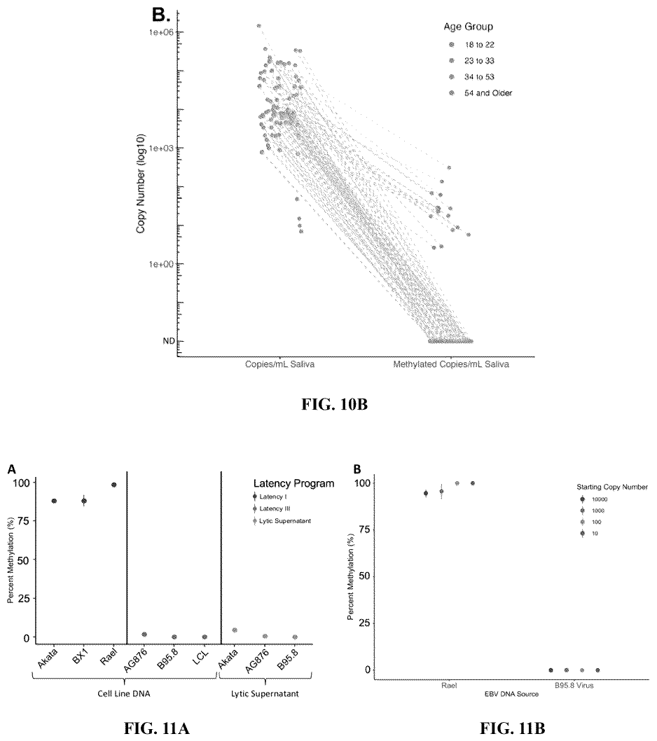

Methylation index was quantified with MSPCP, which was then used to calculate methylated copies per mL of saliva. There was no relationship to amount of EBV in the saliva and the size of its methylated fraction (FIGS. 10A-10B).

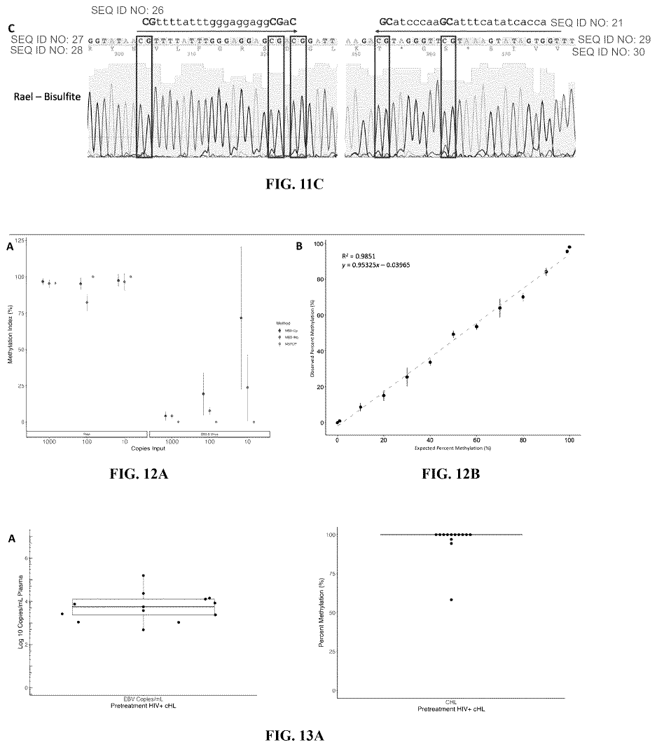

MSPCP specifically amplifies methylated EBV DNA with low minimum input requirement (FIGS. 11A-11C). MSPCP quantifies high percent methylation at Cp in Latency I cell lines, but not Latency III cell lines. When analyzing supernatant DNA from cell lines after lytic induction, the supernatant DNA is largely unmethylated. Experiments performed in triplicate via qPCR. MSPCP reproducibly quantifies Cp methylation in as few as 10 starting input copies of EBV DNA. Experiments performed in triplicate via qPCR. Bisulfite sanger sequencing of Rael cell DNA orthogonally confirms qPCR results that Cp is densely methylated in Latency I. Arrows represent forward and reverse primer sequence targets. CpG sites targeted by MSPCP are highlighted in red boxes.

MSPCP is more sensitive than an alternative bead-based method of quantifying EBV DNA methylation (FIGS. 12A-12B). MSPCP was compared to MBD-Biotin bead capture of methylated EBV DNA. MSPCP was more sensitive than MBD beads targeting either the EBV Wp or Cp. MSPCP quantifies EBV DNA methylation with high specificity in heterogenous samples. All experiments were performed in triplicate. Bars represent standard deviation of replicates.

MSPCP can differentiate cell-derived EBV DNA from virion DNA via C promoter methylation quantification. This method can detect as low as 1% methylation in samples with heterogenous methylation. In an asymptomatic population, approximately 42% of individuals have detectable EBV DNA in their saliva. Saliva copy number can range extensively between individuals, with no relationship to either age or sex. 15 out of 82 asymptomatic individuals have a small methylated fraction of saliva EBV DNA, comprising <2% of EBV DNA molecules, showing no relationship with raw copy number.

Previous research has demonstrated saliva biomarkers that can detect and monitor diseases far from the oropharynx. Saliva EBV DNA may also serve as such a biomarker, but high background EBV levels in healthy individuals confound assays that analyze EBV copy number. Detecting methylated EBV DNA may filter the signal of disease from a high background. Described herein is the disclosure that despite high variance in copy number in asymptomatic individuals, all samples had minimal methylation of saliva EBV DNA, with the majority being devoid of methylation.

MSPCP is a fast and accessible method for quantifying methylated EBV DNA in liquid biopsy samples. Current investigations explore EBV copy number and methylation in pediatric Burkitt lymphoma saliva samples. Other ongoing investigations focus on EBV methylation in plasma samples of low copy number samples.

EBV C promoter methylation can be quantified with high sensitivity and specificity using a methylation-specific PCR assay.

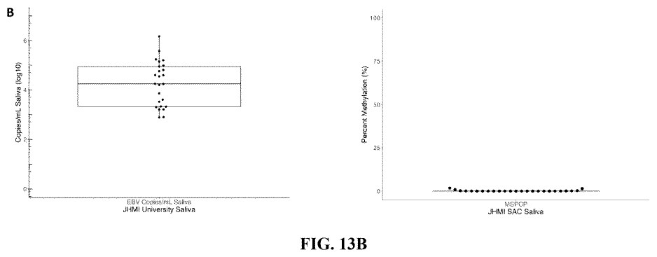

MSPCP has a lower input requirement and higher sensitivity than a bead-based method used for EBV DNA methylation quantification. MSPCP can quantify methylation in plasma, saliva, and formalin-fixed paraffin-embedded tissues. Cp methylation may be useful in identifying EBV(+) cancers (FIGS. 13A-13B).

### Sequences

SEQ ID NO: 1:
CCCAACACTCCACACC
SEQ ID NO: 2:
TCTTAGGAGCTGTCCGAGGG
SEQ ID NO: 3:
FAM/CACACACTACACACACCCACCCGTCTC/3BHQ_1/-3
SEQ ID NO: 4:
CGTTTTATTTGGGAGGAGCGAC
SEQ ID NO: 5:
ACCACTATACTTTACGAACCCTACG
SEQ ID NO: 6:
GAATAATGTTTTATTTGGGAGGAGTGAT
SEQ ID NO: 7:
AAACCACTATACTTTACAAACCCTACA
SEQ ID NO: 8:
TGTAAAACGACGGCCAGTGTTGAGAGGTTAGTGTTTTAAA
TATGTATTTTAGG
SEQ ID NO: 9:
CAGGAAACAGCTATGACCTAAAACCCCTTTACCCAACCC
SEQ ID NO: 10:
ACCTTGTTGGCGGGAGAAGGAATAACGTTTTATTTGGGAGG
AGCGACGGATTATAGCCAATAAGAGAGCTCAAGACGTAGGGT
TCGTAAAGTATAGTGGTTTCGTGGGACCTTAGAGGTGGAGCA
SEQ ID NO: 11:
ACCTTGTTGGCGGGAGAAGGAATAATGTTTTATTTGGGAG
GAGTGATGGATTATAGCCAATAAGAGAGCTCAAGA
TGTAGGGTTTGTAAAGTATAGTGGTTTCGTGGGACCTT
AGAGGTGGAGCA
SEQ ID NO: 12:
GGAGAAGGAATAACGCCTTATCT
SEQ ID NO: 13:
CCACTATACTTTGCGAGCCC
SEQ ID NO: 14:
TGTAAAACGACGGCCAGTTGTTATAAGATTATTAAGT
TGGTGTAAA
SEQ ID NO: 15:
CAGGAAACAGCTATGACCTTTACAACAAAACACAAAA
TTTTTATAA

### REFERENCES

1. Cohen, J. I., Epstein-Barr virus infection. N Engl J Med, 2000. 343(7): p. 481-92.
2. Kimura, H. and J. I. Cohen, Chronic Active Epstein-Barr Virus Disease. Frontiers in Immunology, 2017. 8.
3. Tsao, S. W., et al., The role of Epstein-Barr virus in epithelial malignancies. J Pathol, 2015. 235(2): p. 323-33.
4. El-Sharkawy, A., L. Al Zaidan, and A. Malki, Epstein-Barr Virus-Associated Malignancies: Roles of Viral Oncoproteins in Carcinogenesis. Front Oncol, 2018. 8: p. 265.
5. Hjalgrim, H., et al., Characteristics of Hodgkin's lymphoma after infectious mononucleosis. N Engl J Med, 2003. 349(14): p. 1324-32.
6. Bibas, M. and A. Antinori, EBV and HIV-Related Lymphoma. Mediterr J Hematol Infect Dis, 2009. 1(2): p. e2009032.
7. Castillo, J. J., et al., EBV-positive diffuse large B-cell lymphoma of the elderly: 2016 update on diagnosis, risk-stratification, and management. Am J Hematol, 2016.91(5): p. 529-37.
8. Moormann, A. M., C. J. Snider, and K. Chelimo, The company malaria keeps: how co-infection with Epstein-Barr virus leads to endemic Burkitt lymphoma. Curr Opin Infect Dis, 2011. 24(5): p. 435-41.
9. Chêne, A., et al., A Molecular Link between Malaria and Epstein-Barr Virus Reactivation. PLOS Pathogens, 2007. 3(6): p. e80.
10. Shannon-Lowe, C., A. B. Rickinson, and A. I. Bell, Epstein-Barr virus-associated lymphomas. Philosophical transactions of the Royal Society of London. Series B, Biological sciences, 2017. 372(1732): p. 20160271.
11. Shannon-Lowe, C. and A. Rickinson, The Global Landscape of EBV-Associated Tumors. Frontiers in Oncology, 2019. 9.
12. Tsao, S. W., C. M. Tsang, and K. W. Lo, Epstein-Barr virus infection and nasopharyngeal carcinoma. Philos Trans R Soc Lond B Biol Sci, 2017. 372(1732).
13. Tavakoli, A., et al., Association between Epstein-Barr virus infection and gastric cancer: a systematic review and meta-analysis. BMC Cancer, 2020. 20(1): p. 493.
14. Becnel, D., et al., Pathogenic Role of Epstein-Barr Virus in Lung Cancers. Viruses, 2021. 13(5).
15. Kanda, T., EBV-Encoded Latent Genes, in Human Herpesviruses, Y. Kawaguchi, Y. Mori, and H. Kimura, Editors. 2018, Springer Singapore: Singapore. p. 377-394.
16. Münz, C., Latency and lytic replication in Epstein-Barr virus-associated oncogenesis. Nature Reviews Microbiology, 2019. 17(11): p. 691-700.
17. Mckenzie, J. and A. El-Guindy, Epstein-Barr Virus Lytic Cycle Reactivation, in Epstein Barr Virus Volume 2: One Herpes Virus: Many Diseases, C. Münz, Editor. 2015, Springer International Publishing: Cham. p. 237-261.
18. Kang, M. S. and E. Kieff, Epstein-Barr virus latent genes. Exp Mol Med, 2015. 47(1): p. e131.
19. Niedobitek, G., et al., Heterogeneous Expression of Epstein-Barr Virus Latent Proteins in Endemic Burkitt's Lymphoma. Blood, 1995. 86(2): p. 659-665.
20. Dickerson, S. J., et al., Methylation-Dependent Binding of the Epstein-Barr Virus BZLF1 Protein to Viral Promoters. PLOS Pathogens, 2009. 5(3): p. e1000356.
21. Wille, C. K., et al., Restricted TET2 Expression in Germinal Center Type B Cells Promotes Stringent Epstein-Barr Virus Latency. Journal of Virology, 2017. 91(5): p. e01987-16.
22. Wille, C. K., et al., 5-hydroxymethylation of the EBV genome regulates the latent to lytic switch. Proceedings of the National Academy of Sciences, 2015. 112(52): p. E7257-E7265.
23. Zhang, L., R. Wang, and Z. Xie, The roles of DNA methylation on the promotor of the Epstein-Barr virus (EBV) gene and the genome in patients with EBV-associated diseases. Applied Microbiology and Biotechnology, 2022. 106(12): p. 4413-4426.
24. Marques-Piubelli, M. L., et al., Epstein-Barr virus-associated B-cell lymphoproliferative disorders and lymphomas: a review. Pathology, 2020. 52(1): p. 40-52.
25. Küppers, R., B cells under influence: transformation of B cells by Epstein-Barr virus. Nature Reviews Immunology, 2003. 3(10): p. 801-812.
26. Sun, K., et al., EBV-Positive Gastric Cancer: Current Knowledge and Future Perspectives. Front Oncol, 2020. 10: p. 583463.
27. Sugiura, M., et al., Transcriptional analysis of Epstein-Barr virus gene expression in EBV-positive gastric carcinoma: unique viral latency in the tumour cells. Br J Cancer, 1996. 74(4): p. 625-31.
28. Tao, Q., et al., The Epstein-Barr virus major latent promoter Qp is constitutively active, hypomethylated, and methylation sensitive. J Virol, 1998. 72(9): p. 7075-83.
29. Ambinder, R. F., K. D. Robertson, and Q. Tao, DNA methylation and the Epstein-Barr virus. Seminars in Cancer Biology, 1999. 9(5): p. 369-375.
30. Young, L. S. and C. W. Dawson, Epstein-Barr virus and nasopharyngeal carcinoma. Chin J Cancer, 2014. 33(12): p. 581-90.
31. Shamay, M., N. Hand, and R. F. Ambinder, CpG methylation as a tool to characterize cell-free Epstein-Barr virus DNA. Infectious Agents and Cancer, 2012. 7(1): p. P29.
32. Lam, W. K. J., et al., Methylation analysis of plasma DNA informs etiologies of Epstein-Barr virus-associated diseases. Nature Communications, 2019. 10(1): p. 3256.
33. Shamay, M., et al., CpG methylation in cell-free Epstein-Barr virus DNA in patients with EBV-Hodgkin lymphoma. Blood Advances, 2020. 4(8): p. 1624-1627.
34. Herman, J. G., et al., Methylation-specific PCR: a novel PCR assay for methylation status of CpG islands. Proc Natl Acad Sci USA, 1996. 93(18): p. 9821-6.
35. Tierney, R. J., et al., Methylation of transcription factor binding sites in the Epstein-Barr virus latent cycle promoter Wp coincides with promoter down-regulation during virus-induced B-cell transformation. J Virol, 2000. 74(22): p. 10468-79.
36. Halper-Stromberg, E., et al., CloneRetriever: An Automated Algorithm to Identify Clonal B and T Cell Gene Rearrangements by Next-Generation Sequencing for the Diagnosis of Lymphoid Malignancies. Clin Chem, 2021. 67(11): p. 1524-1533.
37. Kanakry, J. A., et al., Plasma Epstein-Barr virus DNA predicts outcome in advanced Hodgkin lymphoma: correlative analysis from a large North American cooperative group trial. Blood, 2013. 121(18): p. 3547-3553.
38. Lu, J., et al., PrimerSuite: A High-Throughput Web-Based Primer Design Program for Multiplex Bisulfite PCR. Scientific Reports, 2017. 7(1): p. 41328.
39. Kanda, T., et al., Production of high-titer Epstein-Barr virus recombinants derived from Akata cells by using a bacterial artificial chromosome system. J Virol, 2004. 78(13): p. 7004-15.
40. Tao, Q., et al., Epstein-Barr Virus (EBV) in Endemic Burkitt's Lymphoma: Molecular Analysis of Primary Tumor Tissue. Blood, 1998. 91(4): p. 1373-1381.
41. Tierney, R. J., et al., Epstein-Barr virus BamHI W repeat number limits EBNA2/EBNA-LP coexpression in newly infected B cells and the efficiency of B-cell transformation: a rationale for the multiple W repeats in wild-type virus strains. J Virol, 2011. 85(23): p. 12362-75.
42. Liu, M. C., et al., Sensitive and specific multi-cancer detection and localization using methylation signatures in cell-free DNA. Ann Oncol, 2020. 31(6): p. 745-759.
43. Chan, K. C. A., et al., Analysis of Plasma Epstein-Barr Virus DNA to Screen for Nasopharyngeal Cancer. New England Journal of Medicine, 2017. 377(6): p. 513-522.
44. Chan, K. C. A., et al., Plasma Epstein-Barr Virus DNA and Risk of Future Nasopharyngeal Cancer. NEJM Evid, 2023. 2(7): p. EVIDoa2200309.
45. Pisanic, T. R., 2nd, et al., DREAMing: a simple and ultrasensitive method for assessing intratumor epigenetic heterogeneity directly from liquid biopsies. Nucleic Acids Res, 2015. 43(22): p. e154.
46. Fackler, M. J. and S. Sukumar, Quantitation of DNA Methylation by Quantitative Multiplex Methylation-Specific PCR (QM-MSP) Assay. Methods Mol Biol, 2018. 1708: p. 473-496.
47. Fackler, M. J., et al., Development of an automated liquid biopsy assay for methylated markers in advanced breast cancer. Cancer Res Commun, 2022. 2(6): p. 391-401.
48. Zheng, X. H., et al., Saliva biopsy: Detecting the difference of EBV DNA methylation in the diagnosis of nasopharyngeal carcinoma. Int J Cancer, 2023. 153(4): p. 882-892.
49. Mabuchi, S., et al., Role of Epstein-Barr Virus C Promoter Deletion in Diffuse Large B Cell Lymphoma. Cancers (Basel), 2021. 13(3).
50. Young, L. S. and A. B. Rickinson, Epstein-Barr virus: 40 years on. Nature Reviews Cancer, 2004. 4(10): p. 757-768.
51. Shannon-Lowe, C., A. B. Rickinson, and A. I. Bell, Epstein-Barr virus-associated lymphomas. Philosophical transactions of the Royal Society of London. Series B, Biological sciences, 2017. 372(1732): p. 20160271.
52. Zhang, L., R. Wang, and Z. Xie, The roles of DNA methylation on the promotor of the Epstein-Barr virus (EBV) gene and the genome in patients with EBV-associated diseases. Applied Microbiology and Biotechnology, 2022. 106(12): p. 4413-4426.
53. Paulson, E. J. and S. H. Speck, Differential methylation of Epstein-Barr virus latency promoters facilitates viral persistence in healthy seropositive individuals. J Virol, 1999. 73(12): p. 9959-68.
54. Lieberman, P. M., Keeping it quiet: chromatin control of gammaherpesvirus latency. Nature reviews. Microbiology, 2013. 11(12): p. 863-875.
55. Herman, J. G., et al., Methylation-specific PCR: a novel PCR assay for methylation status of CpG islands. Proc Natl Acad Sci USA, 1996.93(18): p. 9821-6.
56. Gandhi, M. K., et al., Plasma Epstein-Barr Virus (EBV) DNA Is a Biomarker for EBV-Positive Hodgkin's Lymphoma. Clinical Cancer Research, 2006. 12(2): p. 460-464.
57. Lam, W. K. J., et al., Methylation analysis of plasma DNA informs etiologies of Epstein-Barr virus-associated diseases. Nature Communications, 2019. 10(1): p. 3256.
58. Mollbrink, A., et al., Monitoring EBV DNA in saliva for early diagnosis of EBV reactivation in solid tumour patients after allogeneic haematopoietic SCT. Bone Marrow Transplant, 2009. 44(4): p. 259-61.

Although the invention has been described with reference to the above examples, it will be understood that modifications and variations are encompassed within the spirit and scope of the invention. Accordingly, the invention is limited only by the following claims.

What is claimed is:

1. A method of identifying a viral nucleic acid sequence in a sample as virion DNA or tumor-derived viral DNA comprising determining a DNA methylation status of the viral nucleic acid sequence, wherein methylation of the viral nucleic acid sequence is indicative of tumor-derived viral DNA and the absence of methylation of the viral nucleic acid sequence is indicative of virion DNA,
thereby identifying the viral nucleic acid sequence as virion DNA or tumor-derived viral DNA.

2. The method of claim 1, wherein the viral nucleic acid sequence is an Epstein-Barr virus (EBV) nucleic acid sequence, optionally wherein the EBV nucleic acid sequence comprises an EBV C promoter (Cp), an EBV W promoter (Wp), EBV Q promoter (Qp), or an EBV F promoter (Fp).

3. The method of claim 2, wherein EBV nucleic acid sequence comprises an EBV Cp.

4. The method of claim 1, wherein determining the DNA methylation status of the viral nucleic acid sequence comprises determining the methylation status of EBV Cp using primers targeting CpG islands in EBV Cp.

5. The method of claim 4, wherein the CpG islands are located within coordinates 11038-11117 of EBV Type 1 reference genome NC_007605.1.

6. The method of claim 4, wherein the primers comprise:
a) methylated-CpG targeting (M) primers, optionally wherein the M primers comprise a nucleic acid sequence comprising SEQ ID NO:4 and SEQ ID NO:5; and
b) unmethylated-CpG targeting (U) primers, optionally wherein the U primers comprise a nucleic acid sequence comprising SEQ ID NO:6 and SEQ ID NO:7.

7. The method of claim 1, wherein a methylated status of the viral DNA is indicative of presence of tumor-derived viral DNA and/or tumor-derived viral DNA in the sample and/or wherein an unmethylated status of the viral DNA is indicative of absence of tumor-derived viral DNA and/or tumor-derived viral DNA in the sample.

8. The method of claim 1, wherein the sample comprises less than about 300 EBV copy number, less than about 100 EBV copy number, or about 50 EBV copy number.

9. The method of claim 1, wherein the sample is a tissue biopsy, plasma, saliva, tumor sample and/or blood sample.

10. A method of diagnosing cancer in a subject comprising identifying a viral nucleic acid sequence as tumor-derived viral DNA in a sample from the subject,
wherein identifying the viral nucleic acid sequence as tumor-derived viral DNA comprises determining a DNA methylation status of the viral nucleic acid sequence according to claim 1, and
wherein detecting methylated tumor-derived viral DNA is indicative of presence of cancer is the subject,
thereby diagnosing the subject with cancer.

11. The method of claim 10, wherein the DNA methylation status is indicative of an EBV latency program.

12. The method of claim 10, wherein the viral nucleic acid sequence comprises cell free DNA (cfDNA).

13. The method of claim 10, wherein the cancer is an EBV-related cancer.

14. A kit comprising:
a) methylated-CpG targeting (M) primers, wherein the M primers comprise a nucleic acid sequence comprising SEQ ID NO:4 and SEQ ID NO:5,
b) unmethylated-CpG targeting (U) primers, wherein the U primers comprise a nucleic acid sequence comprising SEQ ID NO:6 and SEQ ID NO:7, and
c) instructions for using the U and M primers for methylation specific PCR on a biological sample.

15. A method of treating cancer in a subject in need thereof comprising
a) identifying a viral nucleic acid sequence as tumor-derived viral DNA in a sample from the subject, wherein identifying the viral nucleic acid sequence as tumor-derived viral DNA comprises determining a DNA methylation status of the viral nucleic acid sequence, wherein detecting methylated tumor-derived viral DNA is indicative of presence of cancer is the subject and wherein an unmethylated status of the viral DNA is indicative of absence of tumor-derived viral DNA in the sample; and
b) administering an anti-cancer treatment to the subject diagnosed with cancer, thereby treating the cancer in the subject.

16. The method of claim 15, wherein the anti-cancer treatment is chemotherapy, radiation therapy, immunotherapy, resection surgery, hormone therapy, stem cell or bone marrow transplant, or a combination thereof.

17. The method of claim 15, wherein the cancer is an EBV-related cancer.

18. The method of claim 17, wherein the EBV-related cancer is a blood cancer or an epithelial cancer.

19. The method of claim 18, wherein the blood cancer is Burkitt lymphoma, diffuse large B cell lymphoma, Hodgkin lymphoma, or NK/T cell lymphoma and wherein the epithelial cancer is gastric carcinoma, or nasopharyngeal carcinoma.
